(19) United States
(12) Patent Application Publication
(10) Pub. No.: US 2012/0316247 A1
(43) Pub. Date: Dec. 13, 2012

(54) PREVENTION AND TREATMENT OF POST-OPERATIVE COGNITIVE DYSFUNCTION (POCD)

(75) Inventors: Zhongcong Xie, Andover, MA (US); Rudolph Tanzi, Hull, MA (US)

(73) Assignee: THE GENERAL HOSPITAL CORPORATION, Boston, MA (US)

(21) Appl. No.: 13/502,969

(22) PCT Filed: Oct. 20, 2010
(86) PCT No.: PCT/US10/53421

§ 371 Date: Aug. 20, 2012

Related U.S. Application Data
(60) Provisional application No. 61/253,210, filed on Oct. 20, 2009.
(60) Provisional application No. 61/253,196, filed on Oct. 20, 2009.

(51) Int. Cl. A61K 31/05; A61P 25/28; A61P 25/00; A61K 31/08; A61P 23/00

(52) U.S. Cl. 514/722; 514/731

(57) ABSTRACT

Disclosed herein is a method for administering compositions. Also disclosed are kits comprising a compound of formula (I) to (V), or an enantiomer, an analog, a derivative, an isomer, prodrug, or a pharmaceutically acceptable salt thereof, for use in temporal proximity to anesthesia. The methods, compositions and kits provided herein can be used for reducing anesthesia-induced neurotoxicity and post-operative cognitive dysfunction (POCD) in a subject.

### CROSS-REFERENCE TO RELATED APPLICATIONS

This International application paragraphs the benefit of priority under 35 U.S.C. §119(e) of U.S. Provisional Application Nos. 61/253,196 and 61/253,210, filed Oct. 20, 2009, the contents of each of which are incorporated herein by reference in their entirety.

### GOVERNMENT SUPPORT

This invention was made with government support under Grant Nos. NS048140, AG029856 and GM088801 awarded by National Institutes of Health (NIH). The government has certain rights in the invention.

### FIELD OF THE INVENTION

The invention relates generally to methods, compositions and kits for use with anesthesia. The methods, compositions and kits of the invention also relate to prevention and/or treatment of post-operative cognitive dysfunction (POCD).

### BACKGROUND OF THE INVENTION

Post-Operative Cognitive Dysfunction (POCD) is a common complication, especially in the elderly, after cardiac or non-cardiac surgery (e.g., hip replacement) with general anesthesia. POCD is a disorder including deterioration in memory, attention and speed of information processing. It can be chronic and devastating for post-operative recovery of patients. There are over 2.5 million such surgical procedures annually in North America with an incidence of POCD of over 30%.

There is now substantial evidence that many elderly patients experience cognitive deterioration postoperatively. In a prospective, randomized trial of general vs epidural anesthesia with sedation for total knee replacement in patients>70 yr of age, cognitive performance, as assessed with psychometric tests, was worse than the preoperative baseline in 4-6% of patients six months after anesthesia and surgery. Another large, prospective, controlled international study demonstrated a cognitive dysfunction in 9.9% of patients three months postoperatively whereas only about 3% of the age-matched controls were similarly impaired. Among patients over 75 yr of age, 14% had a persistent cognitive dysfunction after general anesthesia and surgery.

Further, a recent study by Wilder et al. (1) investigated more than 5,000 children and reported that children who had early exposure to anesthesia were at an increased risk for developing a learning disability. While there is no direct evidence for a causal relationship between anesthesia administration and later learning-related outcomes (3), the risk for the development of a learning disability increases with longer cumulative duration of anesthesia exposure. Another pilot study by Kalkman et al. (2) has also discussed that children who underwent surgery and anesthesia at younger than 2 yr could be at an increased risk of developing a deviant behavior later in life. These findings discuss that anesthesia can be a significant risk factor for later development of a learning disability and deviant behavior.

Yet the neuropathogenesis of POCD remains to be determined. Several other studies have discussed that the commonly used inhalation anesthetics, for example, isoflurane and sevoflurane, can induce apoptosis in brain tissues of neonatal mice, accompanied by neurocognitive dysfunction (4-6). β-Amyloid protein (Aβ), the key component of senile plaques in patients with Alzheimer disease (AD) (7-9), is the hallmark feature of AD-associated dementia and learning or memory dysfunction (reviewed by 10-12). In addition, neuroinflammation and elevation of the proinflammatory cytokine tumor necrosis factor (TNF)-α have also been discussed to be associated with AD-associated dementia and learning or memory dysfunction (13-15). It is yet largely unknown whether there is a potential association of POCD with Aβ and neuroinflammation. This gap in knowledge impedes the development of therapeutic strategies for treatment and prevention of POCD.

Indeed, no adequate treatment options has yet existed for this distressing post-surgical and post-anesthesia event. Even the advent of newer short-acting anesthetic medicines does not alleviate the post-anesthetic effects on the patients. As the incidence of POCD remains high and such disorder is devastating for the post-operative discovery of patients, discovery of neuropathogenesis of POCD and thus therapeutic interventions for POCD would be urgently needed for reducing healthcare cost and improving quality of life management. As such, there is a strong need for simple and effective methods to prevent and/or treat POCD.

### SUMMARY OF THE INVENTION

Aspects of the present invention stem from the discovery that inhalational anesthesia increases brain cell apoptosis accompanied by neuroinflammation and increases Aβ expression in neonatal mice, and that anesthesia-induced neurotoxic effects are enhanced in neonatal mice with a genetic predisposition to neurodegenerative disorder, e.g., Alzheimer dementia. It was also discovered that Aβ accumulation in the brain contributes to POCD. Further, it was discovered that administration of a compound disclosed herein such as 2-aminoethoxydiphenyl borate (2-APB) prior to anesthesia reduces such anesthetic neurotoxicity.

In accordance with the invention, one aspect relates to a method, in which a compound of the invention, e.g., a compound of formula (I) to (V), an analog, a derivative, an isomer, prodrug or a pharmaceutically acceptable salt thereof, can be administered in temporal proximity to anesthesia, e.g., for facilitating the provision of safer general anesthesia. Another aspect of the invention provides a pharmaceutical composition comprising a compound of formula (I) to (V) for use in temporal proximity to anesthesia, e.g., for prevention of POCD. A further aspect is directed to a pharmaceutical composition comprising a compound of formula (I) to (V) for reducing the level of amyloid-β in the brain of a subject, e.g., a mammal. A still another aspect of the invention provides methods for therapeutic treatment of POCD.

Accordingly, one aspect of the present invention provides a method that include administering to a subject an effective amount of a compound of formula (I), or an isomer, prodrug, a derivative, or a pharmaceutically salt thereof, in temporal proximity to administering an anesthetic to a subject, wherein the formula (I) has the structure:

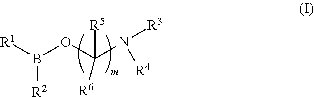

wherein:
R¹ and R² are each independently selected from the group consisting of: F, Br, Cl, I, C₁-C₆ alkyl, C₁-C₆ haloalkyl, C₁-C₆ heteroalkyl, cycloalkyl, aryl or heteroaryl, wherein the alkyl, haloalkyl, heteroalkyl, cycloalkyl, heteroaryl, and aryl can be optionally substituted;
R³ and R⁴ are each independently selected from the group consisting of: hydrogen, F, Br, Cl, I, C₁-C₆ alkyl, C₁-C₆ haloalkyl, C₁-C₆ heteroalkyl, aryl, heteroaryl, cycloalkyl, and heterocycyl, wherein the alkyl, haloalkyl, heteroalkyl, heteroaryl, and aryl can be optionally substituted; or R₃ and R₄ together with the nitrogen to which they are attached form an optionally substituted 5-8 membered cycloalkyl or heterocycyl;
R⁵ and R⁶ are each independently selected from the group consisting of: hydrogen, F, Br, Cl, I, CH₂NR^{3A}R^{4A}, COR^{3A}, COOR^{3A}, C₁-C₁₀ alkyl, C₁-C₁₀ haloalkyl, C₁-C₁₀ heteroalkyl, aryl or heteroaryl, wherein the alkyl, haloalkyl, heteroalkyl, heteroaryl, and aryl can be optionally substituted;
R^{3A} and R^{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted;
m=an integer from 1 and 10;

or an isomer, prodrug, a derivative, or a pharmaceutically acceptable salt thereof.

In certain embodiments, the compound of formula (I) is 2-aminoethoxydiphenyl borate (2-APB), which is represented by formula (II) having the structure:

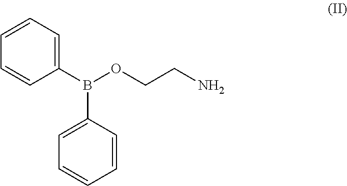

or an isomer, prodrug, a derivative or a pharmaceutically salt thereof.

In some embodiments, the compound of formula (I) (or an isomer, a prodrug or a derivative or a pharmaceutically salt thereof) and the anesthetic can be administered to the subject within one hour of each other. In certain embodiments, the compound of the invention can be administered to the subject prior to, or concurrently with, the anesthetic.

In some embodiments, the anesthetic is halogenated ether anesthetic selected from the group consisting of: isoflurane, enflurane, haloethane, sevoflurane and desflurane. In certain embodiments, the anesthetic is sevoflurane.

In some embodiments, the compound disclosed herein can be administered in an amount effective to decrease or inhibit cognitive impairment in the subject, as compared to absence of administration of the compound. In other embodiments, the compound disclosed herein can be administered in an amount effective to decrease apoptosis and/or the level of amyloid-β in a tissue, e.g., a brain tissue, of the subject, as compared to absence of administration of the compound. In certain embodiments, the effective amount of the compound can be in the range of about 0.1 mg/kg to about 50 mg/kg, or about 1 mg/kg to about 20 mg/kg.

In some embodiments, the subject is at risk of postoperative cognitive dysfunction (POCD), e.g., after administration of the anesthetic. In a particular embodiment, the subject at risk of POCD can be diagnosed with or predisposed to a neurodegenerative disorder, e.g., Alzheimer's disease. In certain embodiments, the subject at risk of POCD is a child or a elderly. In various embodiments, the subject is a mammal, e.g., a human.

Another aspect of the invention relates to a method that include administering an effective amount of a compound of formula (III), including an isomer, a prodrug or a derivative or a pharmaceutically salt thereof, to a subject in temporal proximity to administering an anesthetic to a subject, wherein the formula (III) has the structure:

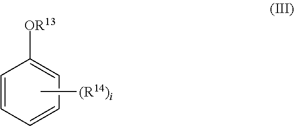

R₁₃ is selected from the group consisting of: hydrogen, CO₂R^{3A}, COR^{3A}, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted;

R₁₄ is selected from the group consisting of: F, Br, Cl, I, CH₂NR^{3A}R^{4A}, SR^{3A}, SO₂R^{3A}, C₁-C₆ alkyl, C₁-C₆ haloalkyl, C₁-C₆ heteroalkyl, C₂-C₆ alkenyl, C₂-C₆ alkynyl, aryl or heteroaryl, NO₂, CF₃, or COR^{3A}, C(OH)R3A, C(NOH)R3A, C(S)R3A, C(OH)(CF3)R3A, C(NOMe)R3A, alkenyl wherein the alkyl, alkenyl, alkynyl, haloalkyl, heteroalkyl, heteroaryl, and aryl can be optionally substituted;

R_{3A} and R_{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted; and

i=0, 1, 2, 3, 4, or 5;

or an isomer, prodrug, a derivative or a pharmaceutically salt thereof.

In certain embodiments, the compound of formula (III) is a γ-aminobutyric acid (GABA) receptor agonist, such as propofol, which is represented by formula (IV) having the structure:

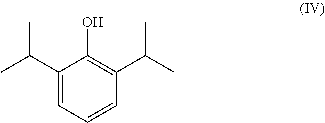

or an isomer, prodrug, a derivative, or a pharmaceutically salt thereof.

In some embodiments, the compound of formula (III) (or an isomer, a prodrug or a derivative or a pharmaceutically salt thereof) and the anesthetic can be administered to the subject within one hour of each other. In certain embodiments, the compound of the invention can be administered to the subject prior to, or concurrently with, the anesthetic.

In some embodiments, the anesthetic is halogenated ether anesthetic selected from the group consisting of: isoflurane, enflurane, haloethane, sevoflurane and desflurane. In certain embodiments, the anesthetic is isoflurane.

In some embodiments, the compound disclosed herein can be administered in an amount effective to decrease or inhibit cognitive impairment in the subject, as compared to absence of administration of the compound. In other embodiments, the compound disclosed herein can be administered in an amount effective to decrease apoptosis and/or the level of amyloid-β in a tissue, e.g., a brain tissue, of the subject, as compared to absence of administration of the compound. In one embodiment, the effective amount of the compound can be in the range of about 1 μM to about 1000 μM, or about 10 μM to about 500 μM.

In some embodiments, the subject is at risk of postoperative cognitive dysfunction (POCD), e.g., after administration of the anesthetic. In a particular embodiment, the subject at risk of POCD can be diagnosed with or predisposed to a neurodegenerative disorder, e.g., Alzheimer's disease. In certain embodiments, the subject at risk of POCD is a child or a elderly. In various embodiments, the subject is a mammal, e.g., a human.

A further aspect of the invention provides a pharmaceutical composition that comprises a compound of formula (I)-(V) for use in preventing POCD. In such embodiments, the composition of the invention can be administered to a subject, e.g., at risk of POCD, in temporal proximity to an anesthetic. Examples of subjects at risk of POCD include, but not limited to, subjects diagnosed with or predisposed to POCD (e.g., genetic condition), a child or a elderly. In various embodiments, the subject is a mammal, e.g., a human.

In yet another aspect, the invention relates to a pharmaceutical composition comprising a compound of formula (I) to (V) for use in reducing the level of amyloid-β in a tissue, e.g., a brain tissue, of a subject. In some embodiments, the subject can be at risk of or diagnosed with POCD. In other embodiments, the subject can be diagnosed with or predisposed to a neurodegenerative disorder, e.g., Alzheimer's disease. In various embodiments, the subject is a mammal, e.g., a human.

In still yet another aspect, the invention provides a method for treating POCD in a subject in need thereof. The method includes (a) selecting the subject that has been diagnosed with POCD, and (b) administering an effective amount of a compound of formula (I), (II), (III), (IV) or (V) to the subject. In some embodiments, the subject with POCD can be diagnosed with or predisposed to a neurodegenerative disorder, e.g., Alzheimer's disease. In various embodiments, the subject is a mammal, e.g., a human.

Kits useful in carrying out the methods described herein also are provided. Such kits comprise at least one container of a compound of formula (I)-(V) and an anesthetic, e.g., sevoflurane or isoflurane, employed in the methods, and optionally contain instructions for the use of the compound of the invention in carrying out the methods described herein.

### BRIEF DESCRIPTION OF THE DRAWINGS

FIGS. 1A to 1E shows that anesthesia with 3% sevoflurane for 6 h induces caspase-3 activation and amyloid precursor protein (APP) processing in the brain tissues of neonatal naïve mice. FIG. 1A is a western blot image indicating that anesthesia with 3% sevoflurane for 6 h (lanes 5-8) induces caspase-3 cleavage (activation) when compared with the control condition (lanes 1-4) in the brain tissues of neonatal naïve mice. FIG. 1B is quantification data of caspase-3 activation determined by the ratio of cleaved (activated) caspase-3 fragment (17-20 kDa) to full length (FL)-caspase-3 (35-40 kDa) in the western blot shown (e.g., in FIG. 1A). Quantification of the western blot shows that 3% sevoflurane anesthesia (black bar) induces caspase-3 activation compared with the control condition (white bar). FIG. 1C is a western blot image showing that sevoflurane anesthesia (lanes 4-6) reduces the levels of APP-C83 and APP-C99 when compared with the control condition (lanes 1-3). FIG. 1D is quantification data of APP-C83 determined by the ratio of APP-C83 to APP-full length (FL) in the western blot (e.g., as shown in FIG. 1C). Quantification of the western blot shows that sevoflurane anesthesia (black bar) decreases the ratio of APP-C83 to APP-FL when compared with the control condition (white bar). FIG. 1E is quantification data of APP-C99 determined by the ratio of APP-C99 to APP-FL in the western blot (e.g., as shown in FIG. 1C). Quantification of the Western blot shows that sevoflurane anesthesia (black bar) decreases the ratio of APP-C99 to APP-FL when compared with the control condition (white bar). The results were averaged from six independent experiments. The symbol “*” indicates a p-value of less than 0.05, while the symbol “**” indicates a p-value of less than 0.01. Color versions of drawings are available in Lu et al., 112 Anesthesiology. 1404 (2010).

FIGS. 2A to 2D shows that anesthesia with 2.1% sevoflurane for 6 h induces caspase-3 activation in the brain tissues of neonatal naïve and Alzheimer disease (AD) transgenic mice. FIG. 2A is a western blot image showing that anesthesia with 2.1% sevoflurane for 6 h (lanes 4-6) induces caspase-3 cleavage (activation) when compared with control condition (lanes 1-3) in the brain tissues of neonatal naïve mice. FIG. 2B is quantification data of caspase-3 activation determined by the ratio of the cleaved (activated) caspase-3 fragment (17-20 kDa) to full length (FL)-caspase-3 (35-40 kDa) in the Western blot (e.g., as shown in FIG. 2A). Quantification of the western blot shows that 2.1% sevoflurane anesthesia (black bar) can still induce caspase-3 activation in the brain tissues of neonatal naïve mice compared with the control condition (white bar). FIG. 2C is a western blot image showing that anesthesia with 2.1% sevoflurane for 6 h (lanes 4-6) induces caspase-3 cleavage (activation) when compared with the control condition (lanes 1-3) in the brain tissues of neonatal AD transgenic mice. FIG. 2D is quantification data of caspase-3 activation determined by the ratio of the cleaved (activated) caspase-3 fragment (17-20 kDa) to full length (FL)-caspase-3 (35-40 kDa) in the Western blot (e.g., as shown in FIG. 2C). Quantification of the Western blot shows that 2.1% sevoflurane anesthesia (black bar) induces caspase-3 activation in the brain tissues of neonatal AD transgenic mice, as compared with the control condition (white bar), normalized to β-actin levels. The results were averaged from 10 independent experiments. The symbol “*” indicates a p-value of less than 0.05, while the symbol “**” indicates a p-value of less than 0.01.

FIGS. 3A and 3B show that anesthesia with 3% sevoflurane for 2 h does not induce caspase-3 activation in the brain tissues of neonatal naïve mice. FIG. 3A is a western blot image showing that anesthesia with 3% sevoflurane for 2 h (lanes 3-5) does not induce caspase-3 cleavage (activation) when compared with the control condition (lanes 1 and 2) in the brain tissues of neonatal naïve mice. FIG. 3B is quantification data of the Western blot (e.g., in FIG. 3A), showing that anesthesia with 3% sevoflurane for 2 h (black bar) does not induce caspase-3 activation compared with the control condition (white bar). The results were averaged from four independent experiments.

FIGS. 4A and 4B show that anesthesia with 3% sevoflurane for 6 h induces a greater degree of caspase-3 activation in the brain tissues of neonatal Alzheimer disease (AD) transgenic mice than that in neonatal naïve mice. FIG. 4A shows a western blot image of caspase-3 activation in the brain tissues of neonatal AD mice and neonatal naïve mice with or without sevoflurane. In FIG. 4A, anesthesia with sevoflurane anesthesia (lanes 2 and 4) induces caspase-3 activation when compared with the control condition (lanes 1 and 3) in naïve mice and AD transgenic mice, respectively. Sevoflurane anesthesia induces a greater degree of caspase-3 activation in AD transgenic mice (lane 4) than in naïve mice (lane 2). FIG. 4B is quantification data of the western blot (e.g., in FIG. 4A), indicating that sevoflurane anesthesia (black bar and hatched bar) induces caspase-3 activation compared with the control condition (white bar and gray bar) in both neonatal naïve and AD transgenic mice, respectively. However, sevoflurane anesthesia induces a greater degree of caspase activation in AD transgenic mice (hatched bar) than in naïve mice (black bar). The results were averaged from four independent experiments. Both the symbols “**” and “##” indicates a p-value of less than 0.01.

FIGS. 5A to 5C show that anesthesia with 3% sevoflurane for 6 h induces more TUNEL-positive cells in the brain tissues of neonatal Alzheimerdisease (AD) transgenic mice than in neonatal naïve mice. FIG. 5A is a representative set of immunostaining images showing that sevoflurane anesthesia (columns 2 and 4) increases TUNEL-positive cells (apoptosis) when compared with the control condition (columns 1 and 3) in the brain tissues of neonatal naïve and AD transgenic mice, respectively. Note that sevoflurane anesthesia causes more TUNEL-positive cells (apoptosis) in neonatal AD transgenic mice (column 4) when compared with neonatal naïve mice (column 2). FIG. 5B is quantification data of the TUNEL image (e.g., in FIG. 5A) showing that sevoflurane anesthesia (black bar and hatched bar) increases TUNEL-positive cells (apoptosis) compared with the control condition (white bar and gray bar) in neonatal naïve mice and AD transgenic mice, respectively. Sevoflurane anesthesia induces more TUNEL-positive cells (apoptosis) in neonatal AD transgenic mice (hatched bar) when compared with neonatal naïve mice (black bar). FIG. 5C is a set of images collected from immuncytochemistry imaging studies, which show that the majority of TUNEL-positive cells (e.g., indicated by the arrows) are neurons detected by positive NeuN staining (original magnification ×200). The results were averaged from five independent experiments. TUNEL=terminal deoxynucleotidyl transferase deoxyuridine triphosphate nick endlabeling. The symbol “*” indicates a p-value of less than 0.01, while the symbol “#” indicates a p-value of less than 0.05.

FIGS. 6A to 6D show that anesthesia with 3% sevoflurane for 6 h increases Aβ levels in the brain tissues of neonatal naïve and Alzheimer disease (AD) transgenic mice. FIG. 6A is a western blot image of Aβ and β-actin levels in the brain tissues of neonatal naïve and AD mice with or without sevoflurane. In FIG. 6A, sevoflurane anesthesia (lanes 3 and 4 and lanes 7 and 8) increases β-amyloid protein (Aβ) levels when compared with the control condition (lanes 1 and 2 and lanes 5 and 6) in the brain tissues of neonatal naïve and AD transgenic mice, respectively. FIG. 6B is quantification data of the Western blot (e.g., in FIG. 6A) showing that sevoflurane anesthesia increases Aβ levels (black bar and hatched bar) when compared with the control condition (white bar and gray bar) in the brain tissues of neonatal naïve mice and AD transgenic mice, respectively. FIG. 6C is the data from sandwich enzyme-linked immunosorbent assay (ELISA) showing that sevoflurane anesthesia increases Aβ42 levels in the brain tissues of AD transgenic mice. FIG. 6C is the data from ELISA sandwich showing that sevoflurane anesthesia does not increase Aβ40 levels in the brain tissues of AD transgenic mice. The results were averaged from six independent experiments. The symbol “*” indicates a p-value of less than 0.05, while the both symbols “**” and “##” indicate a p-value of less than 0.01.

FIGS. 7A to 7D show that 2-Aminoethoxydiphenyl borate (2-APB) (represented by formula II disclosed herein) attenuates sevoflurane-induced caspase-3 activation and Aβ accumulation in the brain tissues of neonatal naïve mice. FIG. 7A is a western blot image of caspase-3 activation in the brain tissues of neonatal naïve mice with or without treatment of 2-APB prior to anesthesia. It shows that anesthesia with 3% sevoflurane for 6 h (lanes 2 and 3) induces caspase-3 cleavage (activation) when compared with the control condition (lane 1), and 2-APB (5 mg/kg: lanes 6 to 8; and 10 mg/kg: lanes 4 and 5) attenuates sevoflurane-induced caspase-3 activation. FIG. 7B is quantification data of the Western blot (e.g., in FIG. 7A), showing that sevoflurane anesthesia (black bar) induces caspase-3 activation compared with the control condition (white bar) and 2-APB (5 mg/kg: gray bar; and 10 mg/kg: hatched bar) attenuates sevoflurane-induced caspase-3 activation (black bar). FIG. 7C is a western blot image of β-amyloid protein (Aβ) level in the brain tissues of neonatal naïve mice with or without treatment of 2-APB prior to anesthesia. It shows that sevoflurane anesthesia (lanes 3 and 4) increases Aβ levels when compared with the control condition (lanes 1 and 2), and 2-APB treatment (lanes 5 and 6) attenuates sevoflurane-induced increases of Aβ levels (lanes 3 and 4). FIG. 7D is quantification data of the Western blot (e.g., in FIG. 7B) showing that sevoflurane anesthesia (black bar) increases Aβ levels compared with the control condition (white bar), and 2-APB treatment (10 mg/kg; hatched bar) attenuates sevoflurane-induced increase of Aβ (black bar). The results were averaged from four independent experiments. The symbols “*” and “#” indicate a p-value of less than 0.05 while the symbols “**” and “##” indicate a p-value of less than 0.01.

FIGS. 8A to 8F show that anesthesia with 3% sevoflurane for 6 h increases tumor necrosis factor (TNF)-α levels in the brain tissues of neonatal Alzheimer disease (AD) transgenic mice. FIG. 8A is a western blot image of TNF-α and β-actin levels in the brain tissues of neonatal AD mice with or without sevoflurane anesthesia. It shows that sevoflurane anesthesia (lanes 5-8) increases TNF-α levels when compared with the control condition (lanes 1-4) in the brain tissues of neonatal AD transgenic mice. FIG. 8B is quantification data of the Western blot (e.g., in FIG. 8A) showing that sevoflurane anesthesia (black bar) increases TNF-α levels compared with the control condition (white bar). FIG. 8C contains data of mRNA level of TNF-α in the brain tissues of neonatal AD mice with or without sevoflurane anesthesia. Sevoflurane anesthesia (black bar) increases messenger ribonucleic acid (mRNA) levels of TNF-α when compared with the control condition (white bar). FIG. 8D is a western blot image of TNF-α and β-actin levels in the brain tissues of neonatal naïve mice with or without sevoflurane anesthesia. It shows that sevoflurane anesthesia (lanes 5-8) does not increase TNF-α levels when compared with the control condition (lanes 1-4) in the brain tissues of neonatal naïve mice. FIG. 8E is quantification of the Western blot (e.g., in FIG. 8D) showing that sevoflurane anesthesia (black bar) does not increase TNF-α levels compared with the control condition (white bar). FIG. 8F contains data of mRNA level of TNF-α in the brain tissues of neonatal naïve mice with or without sevoflurane anesthesia. Sevoflurane anesthesia (black bar) does not increase the mRNA levels of TNF-α when compared with the control condition (white bar) in the brain tissues of neonatal naïve mice. The results were averaged from four independent experiments. The symbol “**” indicates a p-value of less than 0.01.

FIGS. 9A to 9C show the experimental set-up for anesthetic delivery to cells or neurons in vitro. FIG. 9A shows an image of two 6-well plates, in which the cells or neurons are cultured. FIG. 9B shows an image of a sealed plastic box that contain a 6-well plate (as shown in FIG. 9A) stored at a 37° C. incubator. FIG. 9C is an image of anesthesia machine used for anesthetic delivery and Datex infrared gas analyzer for monitoring purpose.

FIGS. 10A to 10B show that propofol (represented by formula IV disclosed herein) attenuates the isoflurane-induced caspase-3 activation in H4-APP cells.

FIG. 10A is a western blot image of caspase-3 activation in the H4-APP cells with or without treatment of propofol prior to isoflurane anesthesia. FIG. 10B is quantification data of the western blot (e.g., in FIG. 10A).

FIGS. 11A to 11B show that propofol does not attenuate the isoflurane-induced caspase-3 activation in H4 naïve cells. FIG. 11A is a western blot image of caspase-3 activation in the H4 naïve cells with or without treatment of propofol prior to isoflurane anesthesia. FIG. 11B is quantification data of the western blot (e.g., in FIG. 11A).

FIGS. 12A to 12B show that propofol does not attenuate the isoflurane-induced caspase-3 activation in primary neurons from the naïve mice. FIG. 12A is a western blot image of caspase-3 activation in the in vitro cultures of primary neurons harvested from the naïve mice with or without treatment of propofol prior to isoflurane anesthesia. FIG. 12B is quantification data of the western blot (e.g., in FIG. 12A).

FIGS. 13A to 13B show the comparison of the propofol effects on isoflurane-induced caspase-3 activation between the H4-APP cells and H4 naïve cells. FIG. 13A is a western blot image of caspase-3 activation in the H4-APP cells and H4 naïve cells with or without treatment of propofol prior to isoflurane anesthesia. FIG. 13B is quantification data of the western blot (e.g., in FIG. 13A).

FIGS. 14A to 14C show that propofol attenuates the isoflurane-induced Aβ oligomerization. FIG. 14A is a western blot image of Aβ40 and Aβ42 levels in the H4-APP cells with or without treatment of propofol prior to isoflurane anesthesia. FIG. 14B is quantification data of Aβ40 levels detected by the western blot (e.g., in FIG. 14A). FIG. 14C is quantification data of Aβ42 levels detected by the western blot (e.g., in FIG. 14A).

### DETAILED DESCRIPTION OF THE INVENTION

Postoperative cognitive dysfunction (POCD) is a cognitive impairment experienced after a clinical intervention, e.g., anesthesia. POCD is a cognitive disorder including deterioration in memory, attention, learning, and speed of information processing. POCD can manifest as short-term symptom, or last for extended periods of time. In some circumstances, POCD can cause a permanent alteration of cognitive functions. Indeed, POCD is commonly observed after anesthesia, e.g., with a halogenated ether such as isoflurane or sevoflurane. However, therapeutic interventions for preventing and treating POCD or safer anesthesia care are lacking.

One aspect of the invention relates to a method in which relates to a method, in which a compound described herein, (e.g., a compound of formula (I) to (V)), or an analog, a derivative, an isomer, prodrug or a pharmaceutically acceptable salt thereof, is administered in temporal proximity to anesthesia, e.g., for facilitating the provision of safer general anesthesia. Another aspect of the invention provides a pharmaceutical composition comprising a compound of formula (I) to (V) for use in temporal proximity to anesthesia, e.g., for prevention of POCD. A further aspect is directed to a pharmaceutical composition comprising a compound of formula (I) to (V) for reducing the level of amyloid-β in the brain of a subject, e.g., a mammal. Another aspect of the invention relates to methods for a therapeutic treatment of POCD.

Another aspect of the invention relates to a method for inhibiting postoperative cognitive dysfunction induced by administration of an anesthetic (e.g., sevoflurane) to a subject. The method comprises administering an effective amount of a compound described herein (e.g., 2-APB or propofol) to the subject in temporal proximity to the administration of the anesthetic, to thereby inhibit the postoperative cognitive dysfunction in the subject induced by the anesthetic.

Another aspect of the invention relates to a method for inhibiting apoptosis and/or Aβ accumulation in the brain cells of a subject induced by administration of an anesthetic (e.g., sevoflurane). The method comprises administering an effective amount of a compound described herein (e.g., 2-APB or propofol) to the subject in temporal proximity to the administration of the anesthetic, to thereby inhibit the apoptosis and/or Aβ accumulation in the brain cells in the subject induced by the anesthetic.

Another aspect of the invention relates to a method for inhibiting caspace-3 activation and/or amyloid precursor protein processing in the brain tissue of a subject induced by administration of an anesthetic (e.g., sevoflurane) to the subject. The method comprises administering an effective amount of a compound described herein (e.g., 2-APB or propofol) to the subject in temporal proximity to the administration of the anesthetic, to thereby inhibit the caspace-3 activation and/or amyloid precursor protein processing in the brain tissue in the subject induced by the anesthetic.

In one embodiment, the methods of the invention are performed on a subject who is determined to be at risk for postoperative cognitive dysfunction, as described herein. In one embodiment, the methods of the invention are performed on a subject who is not found at risk for postoperative cognitive dysfunction. In one embodiment, the methods of the invention are performed on a subject who is diagnosed with or indicated to have an impairment in cognition.

The methods provided herein include administering an effective amount of a compound described herein to a subject in temporal proximity to, for example before, during, and/or after, the administration of such clinical interventions, for example, administration of an anesthetic.

As used herein, “in temporal proximity to” means that the treatments (e.g., administration of a compound disclosed herein and an anesthetic) are administered, in either order, within a specific time of each other. The treatments can be administered within 6 hours of each other, within 5 hours of each other, within 4 hours of each other, within 3 hours of each other, within 2 hours of each other, within 1 hour of each other, within 30 minutes of each other, within 20 minutes of each other, within 10 minutes of each other, within 5 minutes of each other, within 1 minute of each other or substantially simultaneously or concurrently. In one embodiment, the treatments are administered within sufficient time of each other such that the development of POCD induced by the anesthetic is inhibited.

In one embodiment, the compound described herein is administered to the subject prior to the anesthetic, e.g., about 6 hours, about 5 hours, about 4 hours, about 3 hours, about 2 hours, about 1 hour, about 30 minutes, about 20 minutes, about 10 minutes, about 5 minutes, or about 1 minute prior to administration of the anesthetic. In one embodiment, the compound can be administered to the subject about 10 minutes before the anesthetic is administered. In another embodiment, the anesthetic can be administered immediately after administration of a compound described herein. In some embodiments, the compound and the anesthetic can be administered concurrently.

An anesthetic is a drug that causes anesthesia, e.g., which is generally administered to facilitate a surgery, to relieve non-surgical pain or to enable diagnosis of a disease or disorder. An anesthetic can be categorized for use in general anesthesia and local anesthesia. Local anesthesia is a technique to render a region of a subject's body, e.g., a tooth or an area of the skin, insensitive to a sensational feeling, e.g., pain, without affecting cognitive consciousness, while general anesthesia brings a subject to unconsciousness. The methods described herein can be used in temporal proximity to local anesthesia. Exemplary local anesthetics include, but not limited to, aminoesters such as benzocaine, chloroprocaine, cocaine, cyclomethycaine, dimethocaine/Larocaine, piperocaine, propoxycaine, procaine/novocaine, proparacaine, tetracaine/amethocaine; aminoamides such as articaine, bupivacaine, cinchocaine/dibucaine, etidocaine, levobupivacaine, lidocaine/lignocaine, mepivacaine, prilocalne, ropivacaine, trimecaine, lidocaine/prilocalne (EMLA), or natural local anesthetics such as saxitoxin and tetrodotoxin.

The methods described herein can be used in temporal proximity to general anesthesia. With respect to general anesthesia, inhalational anesthetics and intravenous anesthetics can be used with the methods of the invention. Non-limiting examples of inhalational anesthetics include ethers such as diethyl ether, methoxypropane, vinyl ether, halogenated ethers, e.g., desflurane, enflurane, halothane, isoflurane, methoxyflurane; haloalkanes, such as chloroform, halothane, trichloroethylene, cyclopropane, ethylene, nitrous oxide, sevoflurane, xenon, deuterated isoflurane (disclosed in U.S. Pat. No. 4,220,644 and U.S. Pat. No. 4,262,144), hexafluoro-t-butyl-difluoromethyl ether (disclosed in U.S. Pat. No. 3,949,005), deutered analogues of methoxyflurane (disclosed in U.S. Pat. No. 4,281,020), deutered sevoflurane (disclosed in U.S. Pat. No. 5,391,579 and U.S. Pat. No. 5,789,450), and other inhalational anesthetic disclosed in the U.S. Patents, such as U.S. Pat. No. 3,931,344, U.S. Pat. No. 3,932,669, U.S. Pat. No. 3,981,927, U.S. Pat. No. 3,980,714, U.S. Pat. No. 4,346,246, U.S. Pat. No. 3,932,529, U.S. Pat. No. 3,932,667, U.S. Pat. No. 3,954,893, U.S. Pat. No. 3,987,100, U.S. Pat. No. 3,987,203, U.S. Pat. No. 3,995,062, the content of all which is incorporated herein by reference in its entirety. Any of the inhalational anesthetics can be used alone or in combination with other medications to maintain anesthesia. For example, nitrous oxide can be used in combination with other inhalational anesthetics. Accordingly, in some embodiments of the methods, a compound of formula (I) to (V) can be administered to a subject in temporal proximity to at least one or a combination of inhalational anesthetics.

The compound described herein can be administered to a subject in temporal proximity to at least one or a combination of intravenous anesthetics. Examples of intravenous anesthetics include, but not limited to, barbiturates such as hexobarbital, methohexital, narcobarbital, thiopental; opioids such as alfentanil, anileridine, fentanyl, phenoperidine, remifentanil, sufentanil; neuroactive steroids such as alfaxalone and minaxolone; and droperidol, etomidate, fospropofol, gamma-hydroxybutyric acid, ketamine/esketamine, midazolam, propanidid and propofol. Methods for administering to a subject an anesthetic is well known in the art. A skilled practitioner is readily able to determine the amount of an anesthetic administered to each individual based on their weight, age, sex, physical and medical conditions and required length of anesthesia.

Generally, an anesthetic can be re-administered to maintain anesthesia during a course of clinical interventions, such as surgery or diagnosis. Accordingly, in various embodiments, methods of the invention can be repeated after induction of anesthesia. For example, the compound of the invention can be administered in temporal proximity to re-administration of an anesthetic for maintaining anesthesia during a clinical intervention, e.g., a surgery. The anesthetic used for maintaining anesthesia can be same as the anesthetic used for inducing anesthesia. In some cases, an anesthetic used for maintaining anesthesia can be different from the anesthetic used for inducing anesthesia. For instance, a lower concentration of the anesthetic can be used for maintaining anesthesia, as compared to the concentration used for induction of anesthesia. Alternatively, while an intravenous anesthetic can be used to induce anesthesia in a subject, an inhalational anesthetic can be used for anesthesia maintenance. In various embodiments of the methods, the compounds of the invention can be administered to a subject prior to, or concurrently with an anesthetic administered for maintaining anesthesia.

In accordance with the invention, administration of a compound described herein prior to anesthesia, e.g., with sevoflurane or isoflurane, can reduce apoptosis and A3 generation in the brain of mice as demonstrated in the Examples. Accordingly, in some embodiments, methods provided herein can facilitate provision of anesthesia, e.g., inhalational anesthesia, for reducing or inhibiting anesthesia-induced apoptosis and accumulation of Aβ in the brain in a subject.

Additionally, because accumulation of Aβ in the brain of a subject after anesthesia can contribute to POCD as demonstrated in Examples, some embodiments of the methods described herein can lead to prevention or inhibition of postoperative cognitive dysfunction (POCD) such as that induced by administration of an anesthetic. Accordingly, in one aspect, the invention provides a method for prevention or inhibition of POCD in a subject in need thereof. The method comprises administering to the subject a therapeutically effective amount of a compound described herein, e.g., a compound of formula (I)-(V), or an analog, a derivative, an isomer, prodrug, or a pharmaceutically acceptable salt thereof, in temporal proximity to administration of an anesthetic.

Postoperative cognitive dysfunction (POCD) is a cognitive impairment experienced after a clinical intervention, e.g., anesthesia. POCD is a cognitive disorder including deterioration in memory, attention, learning, and speed of information processing. POCD can manifest as short-term symptom, or last for extended periods of time. In some circumstances, POCD can cause a permanent alteration of cognitive functions. Indeed, POCD is commonly observed after anesthesia, e.g., with a halogenated ether such as isoflurane or sevoflurane.

As used herein, the terms “prevent”, “preventing” and “prevention” refer to an complete avoidance of symptoms, such as cognitive impairment or measurable markers of POCD, level of Aβ in the brain. The terms “inhibit”, “inhibiting”, and “inhibition” as used in reference to the development of a disease (e.g., POCD) refer to a reduced severity or degree of any one or more of those symptoms or markers, relative to those symptoms or markers arising in a control or non-treated individual with a similar likelihood or susceptibility of developing POCD, or relative to symptoms or markers likely to arise based on historical or statistical measures of populations affected by POCD. By “reduced severity” is meant at least about 20% in the severity or degree of a symptom or measurable marker, e.g., level of Aβ in the brain, relative to a control, such as without administration of a compound described herein, e.g., at least about 25%, 30%, 40%, 50%, 60%, 70%, 80%, 90%, 95%, 99% or even 100% (i.e., no or non-detectable level of cognitive impairment or measurable markers, e.g., Aβ level).

Methods for diagnosing POCD in a subject are known in the art. For examples, valid assessment of the patient's preoperative and postoperative cognitive function can be performed to characterize POCD. Typical neuropsychological tests known to a skilled artisan include, but not limited to, tests of verbal comprehension, perceptual organization, executive function (abstraction, problem solving and cognitive flexibility), visual tracking, game performance, psychomotor performance, psychomotor speed, digital symbol substitution, processing speed, dot-connection, flicker-fusion, simple reaction time, choice reaction time and perceptive accuracy. Systems, compositions and methods for psychometric assessment of cognitive recovery after anesthesia disclosed in U.S. Pat. App. No.: US 2009/0281398, the content of which is incorporated by reference in its entirety, can also be used for evaluating a subject after anesthesia for POCD.

Further, the results presented in the Examples herein indicate that the formation of Aβ in the brain contributes to POCD. Accordingly, the level of Aβ can be used as a measurable marker for POCD in a subject. For example, the postoperative level of amyloid β protein in the brain of a subject can be detected and then compared to the preoperative level measured in the same subject or compared to the level measured in a subject without administration of a compound described herein in temporal proximity to an anesthetic. Non-invasive methods for quantitative detection of Aβ in the brain of a subject are known in the art, e.g., detection of amyloid-beta protein in the eye lens, which correlates well to the level in the brain, by methods and apparatuses disclosed in US 2010/0110381 and U.S. Pat. No. 7,641,343, the content of which is incorporated herein by reference in its entirety. Alternatively, Aβ in the brain of a subject can be quantitatively detected by positron emission tomography (PET) imaging using amyloid tracers known in the art, e.g., florbetaben (from Bayer), Pittsburgh Compound B (PIB) or the ones disclosed in U.S. Pat. App. No.: US 2007/0053831, US 2010/0056796, and PCT App. No.: WO 2009/146343, WO 2009/155024, WO 2009/155017, WO 2005/040337, WO 2009/117728, WO 2009/117728. Further, the level of amyloid-beta 40 and 42 in the cerebrospinal fluid collected from a patient can be measured with ELISA known to a skilled artisan. It has been previously discussed that the level of amyloid beta proteins in the cerebrospinal fluid (CSF) is inversely proportioned to the level in the brain because sequestration of amyloid beta proteins in the brain results in a decreased level in CSF. Thus, a low level of CSF amyloid-beta proteins indicates accumulation of amyloid beta proteins in the brain.

Additonally, in some embodiments, positron emission tomography (PET) imaging with 2-[¹⁸F]fluoro-2-deoxy-D-glucose (FDG) can be used for measuring reductions in the cerebral metabolic rate for glucose in the brain. As glucose is the main fuel source of the brain cells, PET-FDG can be used to estimate brain cell death (apoptosis) in the brain. For example, a decrease in the metabolic rate for glucose in the brain after anesthesia can indicate brain cell death induced by anesthesia.

Selection of Subjects

In some embodiments of the aspects described herein, the methods further comprise selecting a subject in need thereof, e.g., the subject at risk of POCD after administration of the anesthetic, prior to administering the compounds of the invention. As demonstrated in Examples the effect of an inhalational anesthetic, e.g., sevoflurane, on cell apoptosis and Aβ accumulation in the brain was further enhanced in the neonatal transgenic Alzheimer dementia mice and administration of compounds disclosed herein can reduce such anesthesia-induced neurotoxic effect. Accordingly, subjects that are diagnosed with or at risk of a neurodegenerative disorder, e.g., Alzheimer disease (AD), or a cognitive impairment are examples of subjects suitable for selection.

Methods for diagnosing Alzheimer's disease are well known in the art. For example, the stage of Alzheimer's disease can be assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) manifests symptoms in demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulia is observed. In stage 7 (severe Alzheimer's disease), speech ability becomes limited to just a few words and intelligible vocabulary may be limited to a single word. A patient can lose the ability to walk, sit up, or smile, and eventually cannot hold up the head.

Other alternative diagnostic methods for AD include, but not limited to, cellular and molecular testing methods disclosed in U.S. Pat. No. 7,771,937, U.S. Pat. No. 7,595,167, US 55580748, and PCT Application No.: WO2009/009457, the content of which is incorporated by reference in its entirety. Additionally, protein-based biomarkers for AD, some of which can be detected by non-invasive imaging, e.g., PET, are disclosed in U.S. Pat. No. 7,794,948, the content of which is incorporated by reference in its entirety.

Genes involved in AD risk are also well known in the art. Such AD “risk genes” increase the risk of developing AD. One example of such AD risk genes is apolipoprotein E-e4 (APOE-e4). APOE-e4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-e4 have an increased risk of developing AD. Those who inherit two copies have an even higher risk, but not a certainty of developing AD. In addition to raising risk, APOE-e4 may tend to make symptoms appear at a younger age than usual. Other AD risk genes in addition to APOE-e4 are well established in the art. Some of them are disclosed in US Pat. App. No.: US 2010/0249107, US 2008/0318220, US 2003/0170678 and PCT Application No.: WO 2010/048497, the content of which is incorporated by reference in its entirety. Genetic tests are well established in the art and are available, for example for APOE-e4. A subject carrying the APOE-e4 allele can, therefore, be identified as a subject at risk of developing AD.

Other risk factors for developing AD and cognitive impairment are well established in the art. Exemplary risk factors that increase the likelihood of developing AD and/or a cognitive dysfunction include, but not limited to age, family history, genetic factors, brain health and general health. For example, most individuals with AD manifestation are 65 years old and older and the risk approximately doubles every five years after the age of 65. Therefore, a subject of 65 years and older can be identified as a subject at risk of developing AD and is amenable to the methods provided herein. A similar age-risk relation exists for the development of a cognitive dysfunction. Likewise, those subjects having a parent, brother or sister, or child with AD or a cognitive dysfunction are at increased risk develop AD or a cognitive dysfunction themselves. The risk increases further if more than one family member has the disease or condition. When diseases or conditions tend to run in families, either heredity or environmental factors or both may play a role.

Additional risk factors for AD and cognitive dysfunction have been established, for example head injury, the quality of heart-head connection, cardiovascular health, and general health. There appears to be a strong link between serious head injury and future risk of AD and/or cognitive dysfunction. A subject with a history of head injury can, therefore, be identified as a subject at risk of developing AD or cognitive dysfunction. Some of the evidence links brain health to heart health as the brain is nourished by one of the body's richest networks of blood vessels. The risk of developing AD or cognitive dysfunction appears to be increased by many conditions that damage the heart or blood vessels. These include high blood pressure, heart disease, stroke, diabetes and high cholesterol. A subject suffering from any of these conditions can, therefore be identified as a subject at increased risk of developing AD or cognitive dysfunction and amenable to the methods provided herein. Additionally, general health condition can be a determinant for the risk of developing AD or a cognitive dysfunction. A subject in bad general health, for example an overweight subject, a heavy drinker or smoker, etc., can be identified as a subject at risk of developing AD and/or a cognitive dysfunction, and thus selected for the methods provided herein.

In further embodiments, subjects with Aβ burden are amenable to the methods described herein. Such subjects include, but not limited to, the ones with Down syndrome, Huntington disease, the unaffected carriers of APP or presenilin gene mutations, and the late onset AD risk factor, apolipoprotein e-E4.

As described earlier, most AD manifestations are present in individuals with an age of 65 or above. Thus, the subject at risk of POCD and amenable to the methods of the invention include a elderly. The term “elderly” as used herein refers to an individual at the age or above 60, or at the age or above 65. In such embodiments, the subject can be diagnosed or indicated to have no Alzheimer disease, while in other embodiments the subject can be diagnosed or indicated to have Alzheimer disease. In some embodiments, the subject can be diagnosed or indicated to have no impairment in cognition, while in other embodiments the subject is diagnosed or indicated to have an impairment in cognition.

In further embodiments, the subject amenable to the methods of the invention includes a child. As demonstrated in Example 1, anesthesia can induce apoptosis and A3 generation in the brains of neonatal naïve mice. Further, previous studies have discussed that children who had early exposure to anesthesia were at an increased risk for developing a cognitive disorder such as a learning disability (1). Accordingly, a child can be selected for administration with a compound disclosed herein in temporal proximity to an anesthetic. The term “a child” as used herein refers to a person between birth and puberty, e.g., between the ages of 0 and 21, or between the ages of 0 and 18.

In another embodiment, the subject is in utero. Administration can be, for example, to the mother, or directly to the fetus.

In some embodiments, the subject selected for the methods described herein can be previously diagnosed with short-term POCD manifestation and is now recovered. In other embodiments, the subject selected for administration of a compound described herein in temporal proximity to an anesthetic can be an individual that has POCD.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

In one embodiment, the subject is a mammal. The mammal can be a human, non-human primate, mouse, rat, dog, cat, horse, or cow, but are not limited to these examples. Mammals other than humans can be advantageously used as subjects that represent animal models of anesethesia-induced neurotoxicity. In addition, the methods and compositions described herein can be employed in domesticated animals and/or pets.

Compounds of Formula (I) to (V)

In some embodiments, the compound as described herein is of formula (I), wherein the formula (I) has the structure:

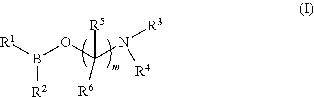

wherein:

R¹ and R² are each independently selected from the group consisting of: F, Br, Cl, I, C₁-C₆ alkyl, C₁-C₆ haloalkyl, C₁-C₆ heteroalkyl, cycloalkyl, aryl or heteroaryl, wherein the alkyl, haloalkyl, heteroalkyl, cycloalkyl, heteroaryl, and aryl can be optionally substituted;

R³ and R⁴ are each independently selected from the group consisting of: hydrogen, F, Br, Cl, I, C₁-C₆ alkyl, C₁-C₆ haloalkyl, C₁-C₆ heteroalkyl, aryl, heteroaryl, cycloalkyl, and heterocycyl, wherein the alkyl, haloalkyl, heteroalkyl, heteroaryl, and aryl can be optionally substituted; or R₃ and R₄ together with the nitrogen to which they are attached form an optionally substituted 5-8 membered cycloalkyl or heterocycyl;

R⁵ and R⁶ are each independently selected from the group consisting of: hydrogen, F, Br, Cl, I, CH₂NR^{3A}R^{4A}, COR^{3A}, COOR^{3A}, C₁-C₁₀ alkyl, C₁-C₁₀ haloalkyl, C₁-C₁₀ heteroalkyl, aryl or heteroaryl, wherein the alkyl, haloalkyl, heteroalkyl, heteroaryl, and aryl can be optionally substituted;

R^{3A} and R^{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted;

m=an integer from 1 and 10;

or an enantiomers, prodrug, a derivative, or a pharmaceutically acceptable salt thereof.

In some embodiments, R¹ and R² can be each independently selected from the group consisting of:

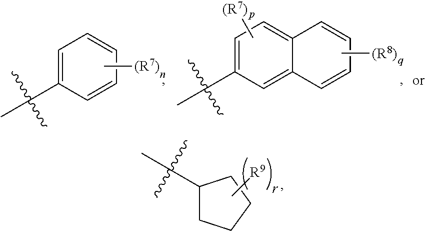

wherein R⁷, R⁸ and R⁹ can be each independently selected from the group consisting of F, Br, Cl, I, OR^{3A}, NR^{3A}R^{4A}, SR^{3A}, SO₂NR^{3A}R^{4A}SO₂R^{3A}, C₁-C₆ alkyl, C₁-C₆ haloalkyl, C₁-C₆ heteroalkyl, C₂-C₆ alkenyl, C₂-C₆ alkynyl, aryl or heteroaryl, NO₂, CF₃, or COR^{3A}, wherein the alkyl, alkenyl, alkynyl, haloalkyl, heteroalkyl, heteroaryl, and aryl can be optionally substituted; n=0, 1, 2, 3, 4, or 5; p=0, 1, 2, or 3; q=0, 1, 2, 3 or 4; r=0, 1, 2, 3, or 4.

In some embodiments, R³ and R⁴ can be each independently selected from the group consisting of: hydrogen, methyl, ethyl, propyl, isopropyl, n-butyl, tert-butyl,

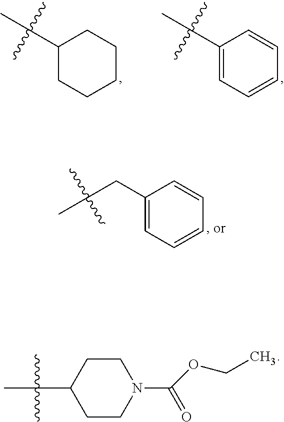

In some embodiments, R₃ and R₄ together with the nitrogen to which they are attached can form an optionally substituted 5-8 membered

cycloalkyl or heterocycyl, e.g.,

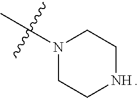

In some embodiments, R⁵ and R⁶ can be each independently selected from the group consisting of: hydrogen, C₁-C₈ alkyl, CO₂CH₃,

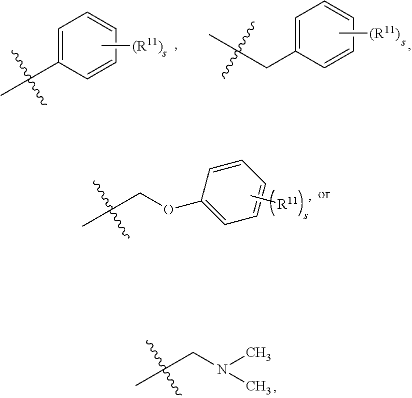

wherein R¹¹ can be selected from the group consisting of: F, Br, Cl, I, C₁-C₆ alkyl, C₁-C₆ haloalkyl, C₁-C₆ heteroalkyl, C₂-C₆ alkenyl, C₂-C₆ alkynyl, aryl or heteroaryl; and s=0, 1, 2, 3, 4, or 5, wherein the alkyl, haloalkyl, heteroalkyl, heteroaryl, and aryl can be optionally substituted.

In some embodiments, the compound of formula (I) can be of formula (V), wherein the formula (V) has the structure:

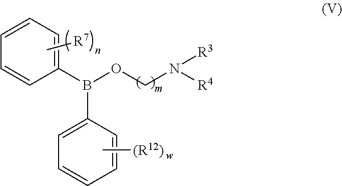

wherein:

R³ and R⁴ are each independently selected from the group consisting of: hydrogen, F, Br, Cl, I, C₁-C₆ alkyl, C₁-C₆ haloalkyl, C₁-C₆ heteroalkyl, aryl, heteroaryl, cycloalkyl, and heterocycyl, wherein the alkyl, haloalkyl, heteroalkyl, heteroaryl, and aryl can be optionally substituted;

R⁷ and R¹² are each independently selected from the group consisting of F, Br, Cl, I, OR^{3A}, NR^{3A}R^{4A}, SR^{3A}, SO₂NR^{3A}R^{4A}SO₂R^{3A}, C₁-C₆ alkyl, C₁-C₆ haloalkyl, C₁-C₆ heteroalkyl, C₂-C₆ alkenyl, C₂-C₆ alkynyl, aryl or heteroaryl, NO₂, CF₃, or COR^{3A}, wherein the alkyl, alkenyl, alkynyl, haloalkyl, heteroalkyl, heteroaryl, and aryl can be optionally substituted;

R^{3A} and R^{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted;

m=an integer between 1 and 10;

n=0, 1, 2, 3, 4 or 5;

w=0, 1, 2, 3, 4 or 5; or an enantiomer, prodrug, a derivative, or a pharmaceutically acceptable salt thereof.

In one embodiment, at least one of n and w (i.e., n only, m only, or both n and m) can be 0.

In another embodiment, n and/or w can be 1, 2, 3, 4, or 5. In such embodiments, R⁷ and R¹² can be each independently selected from the group consisting of: F, Br, Cl, I, OR_{3A} (e.g., O-acryl, —OCH₃), haloalkyl (e.g., CF₃), C₁-C₄ alkyl (e.g., methyl, ethyl, propyl, isopropyl, n-butyl, tert-butyl), C₂-C₄ alkenyl (e.g., —(CH₂)₂), COR_{3A} (e.g., CO-acryl), SO₂R_{3A} (e.g., SO₂N(CH₃)₂).

In some embodiments, R³ and R⁴ can be each independently selected from the group consisting of: hydrogen, methyl, ethyl, propyl, isopropyl, n-butyl, tert-butyl,

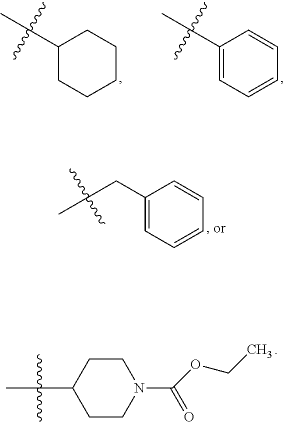

In some embodiments, R₃ and R₄ together with the nitrogen to which they are attached can form an optionally substituted 5-8 membered cycloalkyl or heterocycyl, e.g.,

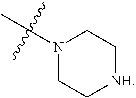

In such embodiment, p can be 1, 2, or 3. In a particular embodiment, p is 2. In one embodiment, at least one of R³ and R⁴ (i.e., R³ only, R⁴ only, or both R³ and R⁴) can be hydrogen.

In one embodiment, the compound of formula (I) is of formula (II), wherein formula (II) has the structure:

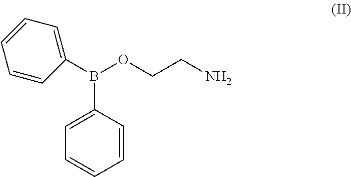

or an enantiomer, prodrug, a derivative, or a pharmaceutically acceptable salt thereof.

The compound of formula (II) is 2-aminoethoxydiphenyl borate (2-APB). 2-APB has been previously discussed for treatment of various diseases, e.g., in U.S. Pat. No. 7,217,701 and U.S. Patent App. No.: US 2002/0107193, (the content of which is incorporated herein by reference in its entirety). However, the prior art does not disclose administration of 2-APB in temporal proximity to anesthesia, or the use thereof for prevention or treatment of POCD.

2-APB is an inositol 1,4,5-trisphosphate (IP₃) receptor antagonist known in the art. Prior art has discussed reducing anesthesia-induced brain cell apoptosis by inhibition of IP3 receptors, e.g., using siRNA or an pharmacological agent against IP3 receptor such as xestospongin C (Yang et al., 109 Anesthesiology. 243 (2008); Wei et al., 108 Anesthesiology. 251 (2008); U.S. Patent App. No.: US 2010/0113570, the content of which are incorporated herein by reference in its entirety). However, these prior-art studies were carried out in vitro, in which the IP3 receptor antagonist was added to the cell culture medium. Thus, the cultured cells could take up the IP3 receptor antagonists directly. However, delivery methods of the IP3 receptor antagonist to the brain in vivo can be completely different. Accordingly, these in vitro experiments does not teach or describe the method for administration of IP3 receptor antagonists in temporal proximity to anesthesia in vivo, e.g., for prevention of POCD. Although Wei and Xie disclosed in 6 Curr. Alzheimer Res. 30 (2009) that intraventricular injection of xestospongin C inhibits isoflurane-induced apoptosis in the developing rat brain, xestospongin C is not a boron compound of formula (I) as disclosed herein. Further, they did not discuss the effect of blocking IP3 receptors on Aβ level, the association of which with POCD, as demonstrated herein in Examples. Accordingly, the use of IP3 receptor antagonists for treatment of POCD is still an unknown.

In some embodiments, a compound of formula (I) used in the methods provided herein can be the compounds disclosed in U.S. Pat. No. 7,217,701, the content of which is incorporated by reference in its entirety. Such exemplary compounds include, but not limited to, 2-cyclohexylaminoethyl bis(3-chloro-4-methylphenyl)borate, 2-aminoethyl bis(4-trifluoromethylphenyl)borate, dicyclopentylborate 2-aminoethyl, 2-aminoethyl bis(4-chloro-2-methylphenyl)borate, 2-aminoethyl bis(4-dimethylaminosulfonylphenyl)borate, 2-aminoethyl bis(2-naphthyl)borate, 2-aminoethyl bis(4-chloro-3-methylphenyl)borate, 2-aminoethyl bis(3-chloro-4-methylphenyl)borate, 2-aminoethyl bis(3,5-dichlorophenyl)borate, 2-dimethylaminoethyl bis(3-chloro-4-methylphenyl)borate, 1-methyl-2-aminoethyl bis(3-chloro-4-methylphenyl)borate, 2-(phenylamino)ethyl bis(3-chloro-4-methylphenyl)borate, 2-(benzylamino)ethyl bis(3-chloro-4-methylphenyl)borate, 2-phenyl-2-aminoethyl bis(3-chloro-4-methylphenyl)borate, 2-(piperazin-1-yl)ethyl bis(3-chloro-4-methylphenyl)borate, 2-(butylamino)ethyl bis(3-chloro-4-methylphenyl)borate, 2-amino-2-(methoxycarbonyl)ethyl bis(4-chlorophenyl)borate, 1-benzyl-2-(methylamino)ethyl bis(3-chloro-4-methylphenyl)borate, 1-phenyl-2-aminoethyl bis(3-chloro-4-methylphenyl)borate, 1-(4-chlorophenoxymethyl)-2-(methylamino)ethyl bis(3-chloro-4-methylphenyl)borate, 1-phenyl-2-(1-(ethoxycarbonyl)piperidin-4-ylamino)ethyl bis(3-chloro-4-methylphenyl)borate, 1-(methylaminomethyl)nonyl bis(3-chloro-4-methylphenyl)borate, 1,2-diphenyl-2-aminoethyl bis(3-chloro-4-methylphenyl)borate, 1-(dimethylaminomethyl)-2-dimethylaminoethyl bis(3-chloro-4-methylphenyl)borate, 2-aminoethyl bis(4-chloro-3-trifluoromethylphenyl)borate, 2-aminoethyl bis(3,5-di(trifluoromethyl)phenyl)borate, 2-aminoethyl bis(3,4,5-trifluorophenyl)borate, 2-aminoethyl bis(2,3,4-trifluorophenyl)borate, 2-aminoethyl bis(3-chloro-4-(1,1-dimethylethyl)phenyl)borate, 2-aminoethyl (4-chlorophenyl)(4-chloro-2-methoxyphenyl)borate, 2-aminoethyl (4-chlorophenyl)(1-naphtyl)borate, 2-aminoethyl (4-chlorophenyl)(1,1′-biphenyl-4-yl)borate, 2-aminoethyl (4-chlorophenyl)(3-chloro-4-phenoxymethylphenyl)borate, 2-aminoethyl (4-chlorophenyl)(4-methylnaphthyl-1-yl)borate, 2-aminoethyl (3-phenylpropyl)phenylborate, 2-aminoethyl (3,3′-diphenylpropyl)phenylborate, 2-aminoethyl (2-phenoxyphenyl)phenylborate, 2-aminoethyl (4-vinylphenyl)(3,4-dichlorophenyl)borate, 2-aminoethyl (4-bromophenyl)(4-chlorophenyl)borate, 2-aminoethyl (4-chlorophenyl)(4-(1,1-dimethylethyl)phenyl)borate, 2-aminoethyl (4-chlorophenyl)(4-iodophenyl)borate, 2-aminoethyl (4-chlorophenyl)(1,1′-biphenyl-2-yl)borate, 2-aminoethyl (3-pyridyl)phenylborate, 2-aminoethyl (3-pyridyl)(4-chlorophenyl)borate, bis[2-[(2-aminoethoxy)phenylboryl]benzyl]ether, bis[4-((2-aminoethoxy)phenylboryl)benzyl]ether, [4-[(2-aminoethoxy)phenylboryl]benzyl][2-[4[(2-aminoethoxy)phenylboryl]phenyl]ethyl]ether, and [2-[(2-aminoethoxy)phenylboryl]benzyl][2-[4[(2-aminoethoxy)phenylboryl]phenyl]ethyl]ether. The U.S. Pat. No. 7,217,701 patent discusses such compounds for inhibiting an increase in intracellular calcium concentration, one of which is 2-APB, but it does not teach or describe the use of such compounds with anesthesia, e.g., for prevention of POCD.

In some embodiments, the compound as described herein can be of formula (III), wherein the formula (III) has the structure:

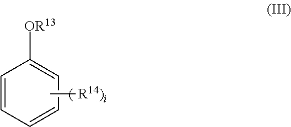

R₁₃ is selected from the group consisting of: hydrogen, CO₂R^{3A}, COR^{3A}, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted;

R₁₄ is selected from the group consisting of: F, Br, Cl, I, CH₂NR^{3A}R^{4A}, SR^{3A}, SO₂R^{3A}, C₁-C₆ alkyl, C₁-C₆ haloalkyl, C₁-C₆ heteroalkyl, C₂-C₆ alkenyl, C₂-C₆ alkynyl, aryl or heteroaryl, NO₂, CF₃, or COR^{3A}, C(OH)R^{3A}, C(NOH)R^{3A}, C(S)R^{3A}, C(OH)(CF₃)R^{3A}, C(NOCH₃)R^{3A}, alkenyl wherein the alkyl, alkenyl, alkynyl, haloalkyl, heteroalkyl, heteroaryl, and aryl can be optionally substituted;

R^{3A} and R^{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl can be optionally substituted;

i=0, 1, 2, 3, 4, or 5; or an enantiomer, prodrug, a derivative, or a pharmaceutically acceptable salt thereof.

In some embodiments, R¹³ can be hydrogen.

In some embodiments, i can be 0.

In some embodiments, i can be 1, 2, 3, 4, or 5. In such embodiments, R¹⁴ can be selected from the group consisting of: methyl, ethyl, isopropyl, n-butyl, isobutyl, sec-butyl, tert-butyl, C₂-C₄ alkenyl, C₂-C₄ alkynyl, C₁-C₄ heteroalkyl (e.g.,

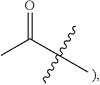

C₁-C₄ haloalkyl, aryl or heteroaryl, wherein the alkenyl, alkynyl, haloalkyl, heteroalkyl, heteroaryl, and aryl can be optionally substituted.

In additional embodiments, R¹⁴ can be selected from the group consisting of:

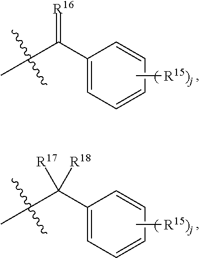

CH₂NR^{3A}R^{4A}, wherein R¹⁴ can be selected from the group consisting of: hydrogen, CF₃, N(CH₃)₂, OCF₃, SO₂ CH₃, SO₂F, SO₂NH₂, CON(CH₃)₂, CONH₂, COCH₃, F, Cl, Br, I, OCH₃, NO₂, C₁-C₆ alkyl, C₁-C₆ haloalkyl, C₁-C₆ heteroalkyl, heteroaryl, or aryl, wherein the alkyl, haloalkyl, heteroalkyl, heteroaryl, and aryl can be optionally substituted; R¹⁶ can be selected from the group consisting of: NOH, S, NOCH₃; R¹⁷ and R¹⁸ can be each independently selected from the group consisting of: hydrogen, OH, CF₃, F, Cl, Br, I. C₁-C₆ alkyl, C₁-C₆ haloalkyl, C₁-C₆ heteroalkyl, heteroaryl, or aryl, wherein the alkyl, haloalkyl, heteroalkyl, heteroaryl, and aryl can be optionally substituted; and j=0, 1, 2, 3, 4 or 5.

In one embodiment, R¹⁴ can be isopropyl.

In one embodiment, the compound of formula (III) can be of formula (IV), wherein the formula (IV) has the structure:

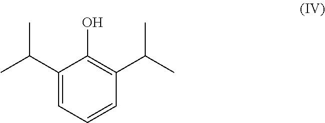

or an enantiomers, prodrug, a derivative, or a pharmaceutically acceptable salt thereof.

In one embodiment, the compound is propofol. Propofol (2,6-diisopropylphenol) is a well-known and widely used intravenous anesthetic agent. It has the advantage of a rapid onset after infusion or bolus injection plus a very short recovery period of several minutes, instead of hours.

Propofol is a γ-aminobutyric acid (GABA) receptor agonist. In some embodiments, without wishing to be bound by the theory, any GABA receptor agonist known in the art (e.g., glutamine) can be used in the methods of the invention.

Propofol is a hydrophobic, water-insoluble oil. It is poorly absorbed in the gastrointestinal tract and only from the small intestine. When orally administered as a homogenous liquid suspension, propofol exhibits an oral bioavailability of less than 5% of that of an equivalent intravenous dose of propofol. Therefore, various propofol prodrugs have been developed to improve propofol adsorption from the gastrointestinal tract and/or minimize first-pass metabolism. Accordingly, in various embodiments, propofol prodrugs that can be used in the methods disclosed herein include, but not limited to, the ones disclosed in the U.S. Pat. No. 7,550,506, U.S. Pat. No. 7,220,875, U.S. Pat. No. 7,230,003, U.S. Pat. No. 7,241,807, and U.S. Pat. App. No.: US 2006/0287525; US 2006/0100160, US 2006/0205969, and PCT Patent App. No.: WO 99/58555, each of which are incorporated by reference herein in its entirety.

Therapeutic uses of propofol prodrugs have been described for treatment of various diseases, such as emesis (US 2007/0259933), metabolic disease, cardiovascular disease, neurodegenerative disorders, liver disease and pulmonary disease (US 2008/0161400), and alcohol withdrawal, anxiety, central pain and pruritis (US 2009/0005444). However, the methods disclosed herein that include administration of a compound, e.g., propofol, in temporal proximity to anesthesia are not described or taught in any of those patents or applications.

In one embodiment, the compound of formula (III) is a propofol analog as described in U.S. Pat. No. 7,586,008 and Krasowski et al. 297. J. Pharmacol. and Experimental Therapeutics. 338 (2001).

Pharmaceutical Compositions and Administration Routes

For administration to a subject, the compound of the invention can be provided in a pharmaceutically acceptable composition. Accordingly, in one aspect, the invention provides a pharmaceutical composition comprising a pharmaceutically effective amount of a compound of formula (I)-(V).

A pharmaceutically acceptable composition comprises a compound of the invention, formulated together with one or more pharmaceutically acceptable carriers (additives) and/or diluents. As described in detail below, the pharmaceutical composition of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C₂-C₁₂ alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” or “effective amount” as used herein means an amount of a compound, material, or composition which is effective for producing some desired therapeutic effect in at least a sub-population of cells in an animal at a reasonable benefit/risk ratio applicable to any medical treatment. For example, an amount of a compound described herein administered to a subject that is sufficient to produce a statistically significant, measurable inhibition of Aβ level in the brain of a subject.

In some embodiments, a compound described herein can be in a therapeutically effective amount of about 0.05 mg/kg to 100 mg/kg of body weight or about 0.1 mg/kg to 50 mg/kg of body weight or about 1 mg/kg to 20 mg/kg of body weight or about 3 mg/kg to 15 mg/kg of body weight, of the subject.

In some embodiments of a compound described herein, a pharmaceutically effective amount can be in the range of about 1 μM to about 1000 μM or about 5 μM to about 800 μM or about 10 μM about 500 μM or about 50 μM to about 250 μM. In some embodiments, the compositions are administered at a dosage so that the compound of the invention has an in vivo, e.g., serum or blood, concentration of less than about 1000 μM, less than about 900 μM, less than about 800 μM, less than about 700 μM, less than about 600 μM, less than about 500 μM, less than about 250 μM, less than about 150 μM, or less than about 100 μM, as determined after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The therapeutically effective dose can be determined by one of ordinary skill in the art, e.g. using cell culture assays. A dose can be formulated in animal models to achieve a circulating plasma concentration range that includes the IC50 (i.e., the concentration of the therapeutic which achieves a half-maximal inhibition of symptoms) as determined in cell culture. An effective dose of a compound described herein can be determined in an animal model by measuring the apoptosis and/or Aβ accumulation in the brain of mice after anesthesia as compared to no administration of the compound. In some embodiments, a dosage comprising a compound described herein is considered to be pharmaceutically effective if the dosage inhibits or decreases apoptosis or Aβ accumulation in the brain by at least about 10%, at least about 15%, at least about 20%, at least about 30%, at least about 40%, at least about 50%, at least about 60%, at least about 70%, at least about 80%, at least about 90%, 95%, 99% or even 100%, as compared to a control (e.g. in the absence of a compound of the invention).

One aspect of the present invention has demonstrated in an in vivo mouse model that a compound of formula (II), e.g., 2-APB is injected intraperitoneally 10 mins before the anesthetic was administered at a pharmaceutically effective amount of about 5 mg/kg or 10 mg/kg to abrogate anesthesia-induced apoptosis and Aβ accumulation in the brains of the mice. Depending on routes of administration, one of skill in the art can determine and adjust an effective dosage of a compound disclosed herein to a subject such as a human subject accordingly, by determining pharmacokinetics and bioavailability of a compound of formula (I) to (V) and analyzing dose-response relationship specific to a compound of the invention in animal models such as a mouse.

Determination of a therapeutically effective amount is well within the capability of those skilled in the art. In various embodiments, the dosage can vary within the range depending upon the dosage form employed and the route of administration utilized. In some embodiments, a therapeutically effective amount of a compound described herein administered to a subject can be dependent upon factors known to a skilled artisan, including bioactivity and bioavailability of the compound (e.g. half-life and stability of the compound in the body), chemical properties of the compound (e.g molecular weight, hydrophobility and solubility); route and frequency of administration, and the like. Further, it will be understood that the specific dose of the pharmaceutical composition comprising a compound as disclosed herein can depend on a variety of factors including physical condition of the subject (e.g. age, gender, weight), and medical history of the subject (e.g. medications being taken, health condition other diseases or disorders). The precise dose of a pharmaceutical composition administered to a subject can be determined by methods known to a skilled artisan such as a pharmacologist, or an anesthesiologist.

As used herein, the term “administer” or “administration” refers to the placement of a composition into a subject by a method or route which results in at least partial localization of the composition at a desired site such that desired effect is produced. Routes of administration suitable for the methods of the invention include both local and systemic administration. Generally, local administration results in more of the composition being delivered to a specific location as compared to the entire body of the subject, whereas, systemic administration results in delivery to essentially the entire body of the subject.

An aggregate or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments of the aspects described herein, the compositions are administered by intravenous infusion or injection.

The amount of a compound described herein that can be combined with a carrier material to produce a single dosage form will generally be that amount of the compound that produces a therapeutic effect. Generally out of one hundred percent, this amount will range from about 0.01% to 99% of the compound, preferably from about 5% to about 70%, most preferably from 10% to about 30%.

In some embodiments, a pharmaceutical formulation comprising a compound of the invention can be administered orally in the form of liquid, syrup, tablet, capsule, powder, sprinkle, chewtab, or dissolvable disc. Alternatively, pharmaceutical formulations of the invention can be administered intravenously or transdermally. For example, in certain embodiments, pharmaceutical compositions comprising a compound of formula (IV), i.e., proprofol, are well known in the art. Accordingly, various pharmaceutical compositions known in the art, e.g., disclosed in U.S. Pat. No. 7,041,705, U.S. Pat. No. 7,097,849, U.S. Pat. No. 5,965,236, for injection, as well as U.S. Pat. App. Publication No.: US 2009/0131514 for buccal and spray formulation, can be used for the purpose of the invention. To overcome any possible solubility problems, the compound can be incorporated with solubilizing agents, surfactants, solvents, or an oil in water emulsion. There are a number of known propofol formulations, such as disclosed in U.S. Pat. Nos. 4,056,635, 4,452,817 and 4,798,846, the contents of which are incorporated herein by reference in its entirety.

With respect to duration and frequency of administration, it is typical for the skilled clinician or anesthesiologist to determine whether to increase or decrease dosage, increase or decrease administration frequency, discontinue administration, resume administration or make other alteration to administration regimen, depending on the administration route and formulations. In some embodiments, the compound described herein is administered at least once in temporal proximity to induction of anesthesia. In further embodiments, the composition described herein can be re-administered at least once, at least twice, at least three times or at least four times during the course of a clinical intervention, e.g., a surgery. Duration of administration can vary with administration route, pharmaceutical composition and a patient's physical and medical condition. For example, intravenous injection of a pharmaceutical composition described herein can last for less than about 1 hour, less than about 45 mins, less than about 30 mins, less than about 20 mins, less than about 15 mins, less than about 10 mins, less than about 5 mins, less than about 30 seconds, less than about 20 seconds, or less than about 5 seconds.

Another aspect of the invention relates to the administration of a compound or pharmaceutical composition described herein to reduce the level of amyloid-β in a tissue, e.g., a brain tissue, of a subject. In one embodiment, the subject is diagnosed with POCD. In one embodiment, the subject is diagnosed with or predisposed to a neurodegenerative disorder, e.g., Alzheimer disease. Methods for diagnosing Alzheimer disease or POCD have been previously described herein. The compound or pharmaceutical composition can be administered to the subject in the absence of anesthesia.

Another aspect of the invention relates to the administration of a compound or pharmaceutical compositions for treatment of POCD. In such embodiments, the method comprises (a) selecting the subject that has been diagnosed with POCD, and (b) administering an effective amount of a compound of formula (I), (II), (III), (IV) or (V) to the subject. In one embodiment, the pharmaceutical composition is administered to the subject in the absence of anesthesia.

Typical methods known to a skilled artisan for diagnosing POCD include neuropsychological assessments, for example, tests of verbal comprehension, perceptual organization, executive function (abstraction, problem solving and cognitive flexibility), visual tracking, game performance, psychomotor performance, psychomotor speed, digital symbol substitution, processing speed, dot-connection, flicker-fusion, simple reaction time, choice reaction time and perceptive accuracy. Systems, compositions and methods for psychometric assessment of cognitive recovery after anesthesia disclosed in U.S. Pat. App. No.: US 2009/0281398, the content of which is incorporated by reference in its entirety, can also be used for evaluating a subject for a risk of POCD after anesthesia.

The terms “treatment” and “treating” as used herein, with respect to treatment of POCD, means preventing the progression of the disease, or altering the course of the disorder (for example, but not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of POCD treatment, therapeutic treatment refers to reducing the cognitive deterioration in a subject and/or inhibiting or reducing the level of Aβ in the brain of a subject that is already inflicted with POCD. Measurable lessening includes any statistically significant decline in a measurable marker or symptom, such as measuring Aβ in the brain by PET scan, or assessing the cognitive improvement with neuropsychological tests such as verbal and perception after treatment.

Kits

Kits that can be used for carrying out the methods described herein also are provided. Such kits contain one or more, typically two or more containers of the components employed in the methods, and optionally contain instructions for the use of the components in carrying out the methods described herein.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the paragraphed invention, because the scope of the invention is limited only by the paragraphs. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. In one embodiment, “reduced”, “reduction” or “decrease” or “inhibit” refers to a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used here in the term “isomer” refers to compounds having the same molecular formula but differing in structure. Isomers which differ only in configuration and/or conformation are referred to as “stereoisomers.” The term “isomer” is also used to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate planepolarized light in different directions). Enantiomers generally have identical physical properties, such as melting points and boiling points, and also have identical spectroscopic properties. Enantiomers can differ from each other with respect to their interaction with plane-polarized light and with respect to biological activity.

The term “analog” as used herein refers to a compound that results from substitution, replacement or deletion of various organic groups or hydrogen atoms from a parent compound. As such, some monoterpenoids can be considered to be analogs of monoterpenes, or in some cases, analogs of other monoterpenoids, including derivatives of monoterpenes. An analog is structurally similar to the parent compound, but can differ by even a single element of the same valence and group of the periodic table as the element it replaces. In one embodiment, the analog exhibits the biological activity of the parent compound as it relates to the method described herein.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound. In one embodiment, the derivative retains the biological activity of the original substance as it relates to the methods described herein.

As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs-principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

As used herein, the term “pharmaceutically-acceptable salts” refers to the conventional nontoxic salts or quaternary ammonium salts of therapeutic agents, e.g., from non-toxic organic or inorganic acids. These salts can be prepared in situ in the administration vehicle or the dosage form manufacturing process, or by separately reacting a therapeutic agent in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The term “alkyl” refers to saturated non-aromatic hydrocarbon chains that may be a straight chain or branched chain, containing 1 to 24 carbon atoms, which may be optionally inserted with N, O, or S. For example, C₁-C₆ indicates that the group may have from 1 to 6 (inclusive) carbon atoms in it. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. The term “alkenyl” refers to an alkyl that comprises at least one double bond. Exemplary alkenyl groups include, but are not limited to, for example, ethylenyl, propylenyl, 1-butenyl, 2-butenyl, isobutylenyl, 1-pentenyl, 2-pentenyl, 3-methyl-1-butenyl, 1-methyl-2-buten-1-yl, 2-methyl-2-butenyl, 2,3-dimethyl-2-butenyl, and the like

The term “alkynyl” refers to an alkyl that comprises at least one triple bond. Exemplary alkynyl groups include, acetylenyl, propynyl, 1-butynyl, 2-butynyl, 1-pentynyl, 2-pentynyl, 3-methyl-1-butynyl, and alkyls and alkenyls with a terminal C≡C.

The term “halogen” refers to any radical of fluorine, chlorine, bromine or iodine.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like. The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 4-nitrophenyl, and the like.

The term “cyclyl” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Representative saturated cyclic alkyls include cyclopropyl, cyclobutyl, cyclopentyl, cyclohexyl, and the like; while unsaturated cyclic alkyls include cyclopentenyl and cyclohexenyl, and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an alkyl group, alkenyl group, and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, or heteroaryl group at any atom of that group. Suitable substituents include, without limitation, halogen, hydroxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The present invention may be defined in any of the following numbered paragraphs:
1. A method comprising administering an effective amount of a compound of formula (I) to a subject in temporal proximity to administering an anesthetic to the subject, wherein the formula (I) has the structure:

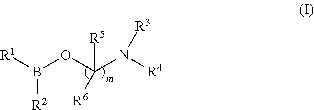

R¹ and R² are each independently selected from the group consisting of: F, Br, Cl, I, C₁-C₆ alkyl, C₁-C₆ haloalkyl, C₁-C₆ heteroalkyl, cycloalkyl, aryl or heteroaryl, wherein the alkyl, haloalkyl, heteroalkyl, cycloalkyl, heteroaryl, and aryl is optionally substituted;
R³ and R⁴ are each independently selected from the group consisting of: hydrogen, F, Br, Cl, I, C₁-C₆ alkyl, C₁-C₆ haloalkyl, C₁-C₆ heteroalkyl, aryl, heteroaryl, cycloalkyl, and heterocycyl, wherein the alkyl, haloalkyl, heteroalkyl, heteroaryl, and aryl is optionally substituted; or R₃ and R₄ together with the nitrogen to which they are attached form an optionally substituted 5-8 membered cycloalkyl or heterocycyl;
R⁵ and R⁶ are each independently selected from the group consisting of: hydrogen, F, Br, Cl, I, CH₂NR^{3A}R^{4A}, COR^{3A}, COOR^{3A}, C₁-C₁₀ alkyl, C₁-C₁₀ haloalkyl, C₁-C₁₀ heteroalkyl, aryl or heteroaryl, wherein the alkyl, haloalkyl, heteroalkyl, heteroaryl, and aryl is optionally substituted;
R^{3A} and R^{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl is optionally substituted;
m=an integer from 1 and 10;
or an enantiomer, prodrug, a derivative or pharmaceutically acceptable salt thereof.
2. The method of paragraph 1, wherein R¹ and R² are each independently selected from the group consisting of:

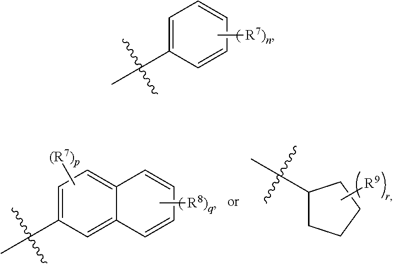

wherein R⁷, R⁸, and R⁹ are each independently selected from the group consisting of F, Br, Cl, I, OR^{3A}, NR^{3A}R^{4A}, SR^{3A}, SO₂NR^{3A}R^{4A} SO₂R^{3A}, C₁-C₆ alkyl, C₁-C₆ haloalkyl, C₁-C₆ heteroalkyl, C₂-C₆ alkenyl, C₂-C₆ alkynyl, aryl or heteroaryl, NO₂, CF₃, or COR^{3A}, wherein the alkyl, alkenyl, alkynyl, haloalkyl, heteroalkyl, heteroaryl, and aryl is optionally substituted; n=0, 1, 2, 3, 4, or 5; p=0, 1, 2, or 3; q=0, 1, 2, 3 or 4; r=0, 1, 2, 3, or 4.
3. The method of paragraph 1, wherein R⁵ and R⁶ are each independently selected from the group consisting of: hydrogen, C₁-C₈ alkyl, CO₂CH₃,

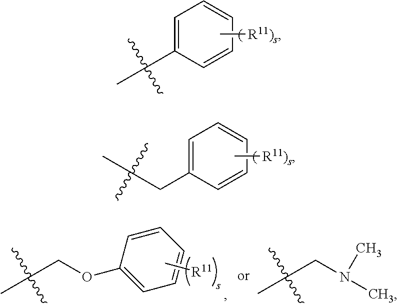

wherein R¹¹ is selected from the group consisting of: F, Br, Cl, I, C₁-C₆ alkyl, C₁-C₆ haloalkyl, C₁-C₆ heteroalkyl, C₂-C₆ alkenyl, C₂-C₆ alkynyl, aryl or heteroaryl; and s=0, 1, 2, 3, 4, or 5, wherein the alkyl, haloalkyl, heteroalkyl, heteroaryl, and aryl is optionally substituted.
4. The method of paragraph 1, wherein R³ and R⁴ are each independently selected from the group consisting of: hydrogen, methyl, ethyl, propyl, isopropyl, n-butyl, tert-butyl,

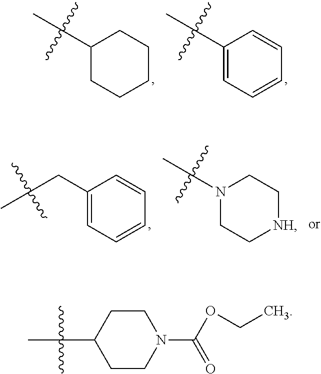

5. The method of paragraph 1, wherein the compound of formula (I) is of formula (II), wherein formula (II) has the structure:

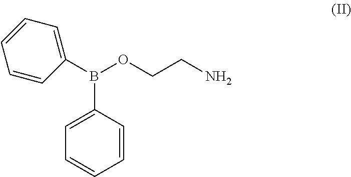

or an enantiomer, prodrug, a derivative or a pharmaceutically acceptable salt thereof.
6. The method of paragraph 1, wherein the compound is 2-aminoethoxydiphenyl borate (2-APB), or an enantiomer, prodrug, a derivative or pharmaceutically acceptable salt thereof, and the anesthetic is sevoflurane.
7. The method of paragraph 1, wherein the subject is at risk for post-operative cognitive dysfunction (POCD) after administration of the anesthetic.
8. The method of paragraphs 1-7, wherein the subject is diagnosed with or predisposed to a neurodegenerative disorder.
9. The method of paragraph 8, wherein the neurodegenerative disorder is Alzheimer's disease.
10. The method of paragraphs 1-9, wherein the subject is a child.
11. The method of paragraphs 1-9, wherein the subject is elderly.
12. The method of paragraphs 1-11, wherein the compound and the anesthetic are administered to the subject within one hour of each other.
13. The method of paragraphs 1-12, wherein the compound is administered to the subject prior to the anesthetic.
14. The method of paragraphs 1-12, wherein the compound is administered to the subject concurrently with the anesthetic.
15. The method of paragraphs 1-14, wherein the effective amount is sufficient to decrease cognitive impairment in the subject resulting from the anesthetic by at least about 20%, as compared to absence of administration of the compound.
16. The method of paragraphs 1-14, wherein the effective amount is sufficient to decrease a level of amyloid-β in a tissue of the subject by at least about 20%, as compared to absence of administration of the compound.
17. The method of paragraphs 1-14, wherein the effective amount is sufficient to decrease apoptosis in a tissue of the subject by at least about 20%, as compared to absence of administration of the compound.
18. The method of paragraphs 16 and 17, wherein the tissue is brain tissue.
19. The method of paragraphs 1-14, wherein the effective amount is from about 0.1 mg/kg to about 50 mg/kg.
20. The method of paragraph 19, wherein the effective amount is from about 1 mg/kg to about 20 mg/kg.
21. The method of paragraphs 1-20, wherein the anesthetic is a halogenated ether anesthetic selected from the group consisting of: isoflurane, enflurane, halothane, sevoflurane and desflurane.
22. The method of paragraph 21, wherein the anesthetic is sevoflurane.
23. The method of paragraphs 1-22, wherein the subject is a mammal.
24. The method of paragraph 23, wherein the mammal is a human.
25. A method comprising administering an effective amount of a compound of formula (III) to the subject in temporal proximity to administering an anesthetic to the subject, wherein the formula (III) has the structure:

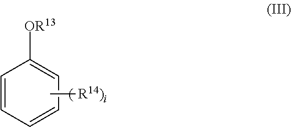

R₁₃ is selected from the group consisting of: hydrogen, CO₂R^{3A}, COR^{3A}, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl is optionally substituted;
R₁₄ is selected from the group consisting of: F, Br, Cl, I, CH₂NR^{3A}R^{4A}, SR^{3A}, SO₂R^{3A} C₁-C₆ alkyl, C₁-C₆ haloalkyl, C₁-C₆ heteroalkyl, C₂-C₆ alkenyl, C₂-C₆ alkynyl, aryl or heteroaryl, NO₂, CF₃, or COR^{3A}, C(OH)R^{3A}, C(NOH)R^{3A}, C(S)R^{3A}, C(OH)(CF₃)R^{3A}, C(NOCH₃)R^{3A}, alkenyl wherein the alkyl, alkenyl, alkynyl, haloalkyl, heteroalkyl, heteroaryl, and aryl is optionally substituted;
R_{3A} and R_{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl is optionally substituted;
i=0, 1, 2, 3, 4, or 5;
or an enantiomer, prodrug, a derivative, or a pharmaceutically acceptable salt thereof.
26. The method of paragraph 25, wherein the compound of formula (III) is a 7-aminobutyric acid (GABA) receptor agonist.
27. The method of paragraph 26, wherein the compound of formula (III) is of formula (IV), wherein formula (IV) has the structure:

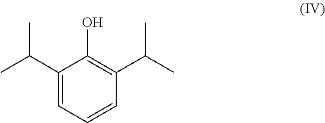

or an enantiomer, prodrug, a derivative or a pharmaceutically acceptable salt thereof.
28. The method of paragraph 25, wherein the compound is propofol, or an enantiomer, prodrug, a derivative or a pharmaceutically acceptable salt thereof, and the anesthetic is isoflurane.
29. The method of paragraphs 25-28, wherein the subject is at risk for post-operative cognitive dysfunction (POCD) after administration of the anesthetic.
30. The method of paragraphs 25-29, wherein the subject is diagnosed with or predisposed to a neurodegenerative disorder.
31. The method of paragraph 30, wherein the neurodegenerative disorder is Alzheimer's disease.
32. The method of paragraphs 25-31, wherein the subject is a child.
33. The method of paragraphs 25-31, wherein the subject at risk of POCD is elderly.
34. The method of paragraphs 25-33, wherein the compound and the anesthetic are administered to the subject within one hour of each other.
35. The method of paragraphs 25-34, wherein the compound is administered to the subject prior to the anesthetic.
36. The method of paragraphs 25-34, wherein the compound is administered to the subject concurrently with the anesthetic.
37. The method of paragraphs 25-36, wherein the effective amount is sufficient to decrease cognitive impairment in the subject resulting from the anesthetic by at least about 20%, as compared to absence of administration of the compound.
38. The method of paragraphs 25-36, wherein the effective amount is sufficient to decrease a level of amyloid-β in a tissue of the subject resulting from the anesthetic by at least about 20%, as compared to absence of administration of the compound.
39. The method of paragraphs 25-36, wherein the effective amount is sufficient to decrease apoptosis in a tissue of the subject resulting from the anesthetic by at least about 20%, as compared to absence of administration of the compound.
40. The method of paragraphs 38 and 39, wherein the tissue is brain tissue.
41. The method of paragraphs 25-36, wherein the effective amount is from about 1 μM to about 1000 μM.
42. The method of paragraph 41, wherein the effective amount is in the range of about 10 μM to about 500 μM.
43. The method of paragraphs 25-42, wherein the anesthetic is a halogenated ether anesthetic selected from the group consisting of: isoflurane, enflurane, halothane, sevoflurane and desflurane.
44. The method of paragraph 43, wherein the anesthetic is isoflurane.
45. The method of paragraphs 25-44, wherein the subject is a mammal.
46. The method of paragraph 45, wherein the mammal is a human.
47. A pharmaceutical composition comprising a pharmaceutically effective amount of a compound of formula (I), (II), (III) or (IV), for use in temporal proximity to administration of an anesthetic to a subject for preventing POCD.
48. The pharmaceutical composition of paragraph 47, wherein the subject is at risk of POCD after administration of the anesthetic.
49. The pharmaceutical composition of paragraphs 47 and 48, wherein the subject is diagnosed with or predisposed to a neurodegenerative disorder.
50. The pharmaceutical composition of paragraph 49, wherein the neurodegenerative disorder is Alzheimer's disease.
51. The pharmaceutical composition of paragraphs 47-50, wherein the subject is a child or elderly.
52. The pharmaceutical composition of paragraphs 47-52, wherein the compound and the anesthetic are administered to the subject within one hour of each other.
53. The pharmaceutical composition of paragraphs 47-52, wherein the compound is administered to the subject prior to the anesthetic.
54. The pharmaceutical composition of paragraphs 47-52, wherein the compound is administered to the subject concurrently with the anesthetic.
55. The pharmaceutical composition of paragraphs 47-54, wherein the pharmaceutically effective amount is sufficient to decrease cognitive impairment in the subject induced by the anesthetic by at least about 20%, as compared to absence of the compound.
56. The pharmaceutical composition of paragraphs 47-54, wherein the pharmaceutically effective amount is sufficient to decrease a level of amyloid-β in a tissue of the subject induced by the anesthetic by at least about 20%, as compared to absence of the compound.
57. The pharmaceutical composition of paragraphs 47-54, wherein the pharmaceutically effective amount is sufficient to decrease apoptosis in a tissue of the subject induced by the anesthetic by at least about 20%, as compared to absence of the compound.
58. The pharmaceutical composition of paragraphs 56 and 57, wherein the tissue is brain tissue.
59. The pharmaceutical composition of paragraphs 47-58, wherein the anesthetic is a halogenated ether anesthetic selected from the group consisting of: isoflurane, enflurane, halothane, sevoflurane and desflurane.
60. The pharmaceutical composition of paragraph 59, wherein the anesthetic is sevoflurane or isoflurane.
61. The pharmaceutical composition of paragraphs 47-60, wherein the subject is a mammal.
62. The pharmaceutical composition of paragraph 61, wherein the mammal is a human.
63. The pharmaceutical composition of paragraphs 47-57, wherein the pharmaceutically effective amount of the compound of formula (I) or (II) is from about 0.1 mg/kg to about 50 mg/kg.
64. The pharmaceutical composition of paragraph 63, wherein the pharmaceutically effective amount of the compound of formula (I) or (II) is from about 1 mg/kg to about 20 mg/kg.
65. The pharmaceutical composition of paragraphs 47-57, wherein the pharmaceutically effective amount of the compound of formula (III) or (IV) is from about 1 μM to about 1000 M.
66. The pharmaceutical composition of paragraph 65, wherein the pharmaceutically effective amount of the compound of formula (III) or (IV) is from about 10 μM to about 500M.
67. A pharmaceutical composition comprising a pharmaceutically effective amount of a compound of formula (I), (II), (III) or (IV) for use, in reducing a level of amyloid-β in a tissue of a subject.
68. The pharmaceutical composition of paragraph 67, wherein the tissue is brain tissue.
69. The pharmaceutical composition of paragraph 67, wherein the subject is at risk of or diagnosed with POCD.
70. The pharmaceutical composition of paragraphs 67, wherein the subject is diagnosed with or predisposed to a neurodegenerative disorder.
71. The pharmaceutical composition of paragraph 70, wherein the neurodegenerative disorder is Alzheimer's disease.
72. The pharmaceutical composition of paragraphs 67-71, wherein the subject is a mammal.
73. The pharmaceutical composition of paragraph 72, wherein the mammal is a human.
74. A method for treating POCD in a subject in need thereof, comprising: (a) selecting the subject that has been diagnosed with POCD, and (b) administering an effective amount of a compound of formula (I), (II), (III) or (IV) to the subject to thereby treat POCD.
75. The method of paragraph 74, wherein the subject with POCD is diagnosed with or predisposed to a neurodegenerative disorder.
76. The method of paragraph 75, wherein the neurodegenerative disorder is Alzheimer's disease.
77. The method of paragraphs 74-76, wherein the subject is a mammal.
78. The method of paragraph 77, wherein the mammal is a human.

The invention is further illustrated by the following examples, which should not be construed as further limiting.

### EXAMPLES

The examples presented herein relate to the use of small-molecule compounds, e.g., 2-aminoethoxydiphenyl borate (2-APB) or 2,6-diisopropylphenol (propofol), prior to administration of anesthesia for inhibiting apoptosis and formation of amyloid-β in vitro and in vivo. In accordance with the invention, in some embodiments, the methods and small molecules described herein can be used for prevention and/or treatment of POCD. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the paragraphs to the invention, but are rather intended to be exemplary of certain embodiments. Any variations in the exemplified methods which occur to the skilled artisan are intended to fall within the scope of the present invention.

### Methods of the Invention

### Examples 1 to 7

Animal Treatments.

The animal protocol was approved by the Standing Committee on Animals at Massachusetts General Hospital (Boston, Mass.). Naive mice (C57BL/6J mice [The JacksonLaboratory, Bar Harbor, Me.]) and Alzheimer's disease (AD) transgenic mice (B6.Cg-Tg[APPswe, PSEN1dE9]85 Dbo/J, [The Jackson Laboratory]) were distinguished by genotyping. All animals (3-12 mice per experiment) were 6 days old at the time of anesthesia and were randomized by weight and gender into experimental groups that received either 3 or 2.1% sevoflurane plus 60% oxygen for either 6 or 2 h, and control groups received 60% oxygen for 6 or 2 h at identical flow rates in identical anesthetizing chambers. Sevoflurane anesthesia was selected because a recent study by Satomoto et al. (6) indicated that anesthesia with 3% sevoflurane plus 60% oxygen for 6 h does not significantly alter blood gas and brain blood flow, which is consistent with our pilot studies. The mortality rate of the mice after the administration of anesthesia with 3% sevoflurane plus 60% oxygen for 6 h in the studies disclosed herein was approximately 10-15%, which could be because of the higher than clinically relevant concentration of sevoflurane. The high concentration of sevoflurane anesthesia was used to illustrate the difference of sevoflurane-induced neurotoxicity between neonatal naïve and AD transgenic mice. Moreover, the effects of anesthesia with 2.1% sevoflurane, a more clinically relevant concentration of sevoflurane (which did not cause the death of the mice), on caspase-3 activation and Aβ levels in the brain tissues of neonatal mice was assessed. Anesthetic and oxygen concentrations were measured continuously (Datex, Tewksbury, Mass.), and the temperature of the anesthetizing chamber was controlled to maintain the rectal temperature of the mice at 37°±0.5° C. In the interaction studies, the inositol trisphosphate receptor (IP3R) antagonist 2-aminoethoxydiphenyl borate (2-APB) (5 and 10 mg/kg) was administered to the mice via intraperitoneal injection 10 min before the anesthetic was administered. 2-APB was first dissolved in dimethyl sulfoxide to 20 μg/μl and then diluted with saline to 0.25 μg/μl (1:80 dilution) and to 0.5 μg/μl (1:40 dilution).

Tissue Preparation.

Immediately after sevoflurane anesthesia, the mouse was decapitated, and the brain cortex was harvested. The brain tissues were homogenized in an immunoprecipitation buffer (10 mM Tris-HCl, pH 7.4, 150 mM NaCl, 2 mM ethylenediaminetetraacetic acid, and 0.5% Nonidet P-40) plus protease inhibitors ([1 μg/ml aprotinin, 1 μg/ml leupeptin, and 1 μg/ml pepstatin A]; [Roche, Indianapolis, Ind.]). The lysates were collected, centrifuged at 13,000 rpm for 15 min, and quantified for total proteins by using the bicinchoninic acid protein assay kit (Pierce, Iselin, N.J.).

Western Blots Analysis.

The harvested brain tissues were subjected to Western blots as described by Xie et al. (16). Briefly, 60 μg of each lysate was separated on sodium dodecyl sulfate polyacrylamide gel electrophoresis gels and transferred to polyvinylidene difluoride blots (Bio-Rad, Hercules, Calif.) using a semidry electrotransfer system (Amersham Biosciences, San Francisco, Calif.). The blot was incubated overnight at 4° C. with primary antibodies, followed by washes and incubation with appropriate secondary antibodies, and visualized with a chemoluminescence system. A caspase-3 antibody (1:1,000 dilution; Cell Signaling Technology, Danvers, Mass.) was used to recognize the caspase-3 fragment (17-20 kDa) resulting from cleavage at aspartate position 175 and fulllength (FL) caspase-3 (35-40 kDa). TNF-α levels were recognized by antibody ab6671 (26 kDa; 1:1,000; Abcam, Cambridge, Mass.). The antibody to nontargeted protein β-actin was used to control for loading differences in total protein amounts. The figures showing blots with only the caspase-3 fragment are the same Western blots with extended exposure time during the development of the film. The signal of the Western blot band was detected using Molecular Imager VersaDoc MP 5000 System (Bio-Rad). The intensity of signals was analyzed using a Bio-Rad image program (Quantity One) and a National Institutes of Health Image Version 1.37 (National Institutes of Health, Bethesda, Md.). Western blots were quantified using two steps. First, levels of β-actin was used to normalize (e.g., determining the ratio of FL caspase-3 amount to β-actin amount) the levels of proteins to control for loading differences in total protein amounts. Second, the changes in the levels of proteins in the mice treated with sevoflurane were presented as the percentage or fold of those in the mice treated with control conditions. One hundred percent or one-fold change in the protein levels described in the Examples refers to the control levels for comparison with experimental conditions.

Immunoblot Detection of Aβ.

The brain samples were homogenized (150 mM NaCl with a protease inhibitor cocktail in 50 mM Tris, pH 8.0) and centrifuged (65,000 rpm for 45 min), and then the supernatant was removed. The pellet was then resuspended by sonication and incubated for 15 min in homogenization buffer containing 1% sodium dodecyl sulfate. After pelleting of insoluble material (18,000 rpm for 15 min), the sodium dodecyl sulfate extract was electrophoresed on sodium dodecyl sulfate polyacrylamide gel electrophoresis (4-12% bis-tris polyacrylamide gel; Invitrogen, Carlsbad, Calif.), blotted to polyvinylidene fluoride membrane, and probed with a 1:200 dilution of Aβ 6E10 (Convance, Berkeley, Calif.) (16,17).

Quantification of Aβ Using a Sandwich Enzyme-linked Immunosorbent Assay.

The Aβ42 and Aβ40 levels in the brain tissues of AD transgenic mice were measured by using sandwich enzyme-linked immunosorbent assay (ELISA). The Human Aβ (1-42) ELISA kit or Human Aβ (1-40) ELISA kit (Wako, Richmond, Va.) was used to detect the levels of Aβ42 or Aβ40, respectively. The monoclonal antibody BAN50, the epitope of which is human Aβ (1-16), was coated on 96-well plates and acted as a capture antibody for the N-terminal portion of human Aβ42 or human Aβ40. Captured human Aβ42 or human Aβ40 was recognized by another antibody BC05 or BA27, which specifically detected the C-terminal portion of Aβ42 or Aβ40, respectively. The 96-well plates were incubated overnight at 4° C. with test samples and control, and then BC05 or BA27 was added. The plates were then developed with tetramethylbenzidine reagent and terminated by stop solution, and well absorbance was measured at 450 nm. Aβ42 and Aβ40 levels in the test samples were determined by comparing the results with signals from the controls using the standard curve. The mouse brain tissue samples were prepared by using the same method in the section of the immunoblot detection of Aβ.

Terminal Deoxynucleotidyl Transferase dUTP Nick End Labeling Staining Assay. Mice were perfused transcardially with 0.1 M phosphate buffer with a pH of 7.4 followed by 4% paraformaldehyde in a 0.1 M phosphate-buffered saline immediately after the administration of anesthesia with 3% sevoflurane plus 60% oxygen for 6 h. The mouse brain tissues were removed and exposed to immersion fixation for 24 h at 4° C. in 4% paraformaldehyde, and then 5-μm paraffin-embedded sections were made from the brain tissues. TMRred kit (Roche, Palo Alto, Calif.) was used for terminal deoxynucleotidyl transferase dUTP nick end labeling (TUNEL) staining. Briefly, the brain sections were incubated in a permeabilization solution and then incubated with a TUNEL reaction mixture. Finally, the sections were incubated with 10 μg/ml Hoechst 33342 in a humidified dark chamber. The sections from the same brain areas between control group mice and the sevoflurane-treated mice were then analyzed in a mounting medium under a fluorescence microscope. The TUNEL-positive cells and total cells in five areas of the brain section from each of the mice in the experiments were counted blind-fold under a 20× objective microscope lens. For the double immunocytochemistry staining to identify the cell type of the TUNEL-positive cells, the mouse brain tissues were quickly removed after sevoflurane anesthesia, put into a container with dry ice and ethanol, and then kept in a −80° C. freezer. Five-micrometer frozen sections were cut using a cryostat. The sections were fixed successively with 100% methanol at −20° C. for 20 min and incubated with permeabilization solution (7.8% gelatin and 1.25 ml saponin [10%] in 500 ml phosphate-buffered saline) for min. The sections were incubated in 10% donkey serum in permeabilization solution for min and then incubated with antibody for NeuN to identify neurons (1:500, mab 377, antimouse; Millipore, Billerica, Mass.), antibody for Glial fibrillary acidic protein to identify astrocytes (1:100, ab16997, antirabbit; Abcam), and antibody for Iba1 to identify microglia cells (1:100, ab5076, antigoat; Abcam) at 4° C. overnight. Then sections were exposed to secondary antibodies Alexa Fluor®488 goat antimouse immunoglobulin G (1:1000; Invitrogen), Alexa Fluor®488 goat antirabbit Immunoglobulin G (1:1000; Invitrogen), and donkey antigoat immunoglobulin G-Cy2 (1:100; Jackson ImmunoResearch Inc., West Grove, Pa.) for 1 h at 37° C. in a dark chamber followed by TUNEL staining. The sections were counterstained with 10 μg/ml Hoechst 33342 at room temperature for 10 min. Finally, the sections were mounted and immediately viewed using a fluorescence microscope.

Reverse Transcriptase Polymerase Chain Reaction.

Real-time reverse transcriptase polymerase chain reaction was carried out using the QuantiTect SYBR Green real-time polymerase chain reaction kit (Qiagen, Valencia, Calif.). TNF-α messenger ribonucleic acid levels were determined and standardized with glyceraldehyde-3-phosphate dehydrogenase as an internal control. Primers of mouse TNF-α (ID No., QT00104006) and mouse glyceraldehyde-3-phosphate dehydrogenase (ID No., QT01658692) were purchased from Qiagen.

Statistics.

Given the potential presence of background caspase-3 activation and apoptosis in the brain tissues of neonatal mice, absolute values were not used to describe changes in caspase-3 activation and apoptosis. Instead, caspase-3 activation and cell apoptosis were presented as a percentage or fold of those in the control group in naïve mice or AD transgenic mice. One hundred percent or onefold caspase-3 activation or apoptosis refers to the control levels for the purposes of comparison with experimental conditions. The changes in the levels of caspase-3 activation, apoptosis, levels of Aβ, and TNF-α in treated mice were presented as percentages or folds of those in mice in the control condition. Data were expressed as mean±SD. The number of samples varied from 3 to 12, and the samples were normally distributed. A randomization table generated using a computer random number generator and stored in a Microsoft Excel spreadsheet was used to randomize animals to conditions. ANOVA or Student t test was used to compare the differences from the control group. Only a single measurement of each outcome value was collected from each experimental animal. As a result, no repeated measurement was involved in the analysis. Post hoc adjustment for multiple comparisons was conducted using the Bonferroni method. P values of less than 0.05 (* or #) and 0.01 (** or ##) were considered statistically significant. The significance testing was two tailed, and SAS software (Cary, N.C.) was used to analyze the data.

### Example 1

### Post-operative cognitive dysfunction and Aβ

Post-operative cognitive dysfunction (POCD), the most common post-operative complications in older adults, can increase peri-operative morbidity, mortality, and cost. However, its neuropathogenesis remains to be determined. This gap in knowledge prevents the development of therapeutic interventions to prevent and treat POCD.

Amyloid-β (Aβ), the key component of senile plaques in Alzheimer's disease (AD) patients, is associated with cognitive dysfunction. However, no previous studies have discussed the potential association of POCD with the level of Aβ. Thus, presented herein is the first evidence to demonstrate the association between pre-operative Aβ levels in cerebrospinal fluid (CSF) with POCD. Cognitive tests were performed approximately one week before the surgery, as well as one week and three months after the surgery, on the subjects either at their home or rehabilitation facility. The verbal learning test and brief visuospatial memory test scores were obtained by using the raw score representing pre-operative baseline minus that representing post-operative ones. A positive value indicates a decline of cognitive function. All of the patients had either total hip replacement or total knee replacement under spinal anesthesia. CSF was obtained from the patients during the administration of the spinal anesthesia immediately before the surgery. The Aβ levels in the collected CSF were measured with ELISA.

The reduced levels of both Aβ40 and Aβ42 levels in CSF were found to be associated with post-operative verbal learning test three month (Table 1), and brief visuospatial memory one week (Table 2), after the surgery, respectively. Linear regression analysis indicated that the subjects who had low pre-operative Aβ40 and Aβ42 levels in CSF performed poorly on the verbal learning test and brief visuospatial memory test (higher score: which value in Table 1 does it refer to?); the subjects who had high pre-operative levels of Aβ40 and Aβ42 in CSF performed well on the tests (lower score: which value in Table 1 does it refer to?).

TABLE 1
Positive correlations of Aβ40 in CSF and verbal learning test.

| | R | P | N |
|---|---|---|---|
| 1 Week after the surgery | −0.22437 | 0.0513 | 76 |
| 3 Months after the | −0.29547 | 0.0257 | 57 |
| surgery | | | |

Levels of Aβ40 correlated significantly with the change in score on verbal learning test at three months post-operative evaluation, but the extent of correlation was lower at one week post-operative evaluation.

TABLE 2
Positive correlations of Aβ42 and brief visuospatial memory test.

| | R | P | N |
|---|---|---|---|
| 1 Week after the Surgery | −0.31964 | 0.007 | 70 |
| 3 Months after the | 0.10521 | 0.449 | 54 |
| Surgery | | | |

Levels of Aβ42 correlated significantly with the change in brief visuospatial memory test score at the one week post-operative evaluations, but did not correlate with the change in score at the three months post-operative evaluation.

These results demonstrate that reduced Aβ levels in CSF are associated with POCD. Since low Aβ levels in CSF can reflect the sequestration of Aβ into brain amyloid plaques, reduced levels of both Aβ40 and Aβ42 levels in CSF indicates that Aβ accumulation in central nervous system (CNS) can be the underlying mechanism of POCD.

### Example 2

### Anesthesia (e.g., Sevoflurane) Induced Caspase-3 Activation and Amyloid Precursor Protein Processing in the Brain Tissues of Neonatal Mice

It has been previously reported that the commonly used inhalation anesthetic, sevoflurane, can cause neurotoxicity by inducing apoptosis and enhancing Aβ levels in vitro and in brain tissues of adult naïve mice (18). But the effects of sevoflurane on apoptosis, Aβ accumulation, and neuroinflammation in neonatal mice remain largely to be determined. Furthermore, the comparison of these effects between neonatal naïve mice and AD transgenic mice has not been discussed. Therefore, the effects of sevoflurane on apoptosis, Aβ accumulation, and neuroinflammation in neonatal (6 days old) naïve (C57BL/6J) and AD transgenic (B6.Cg-Tg[APPswe, PSEN1dE9]85 Dbo/J) mice were assessed as described herein.

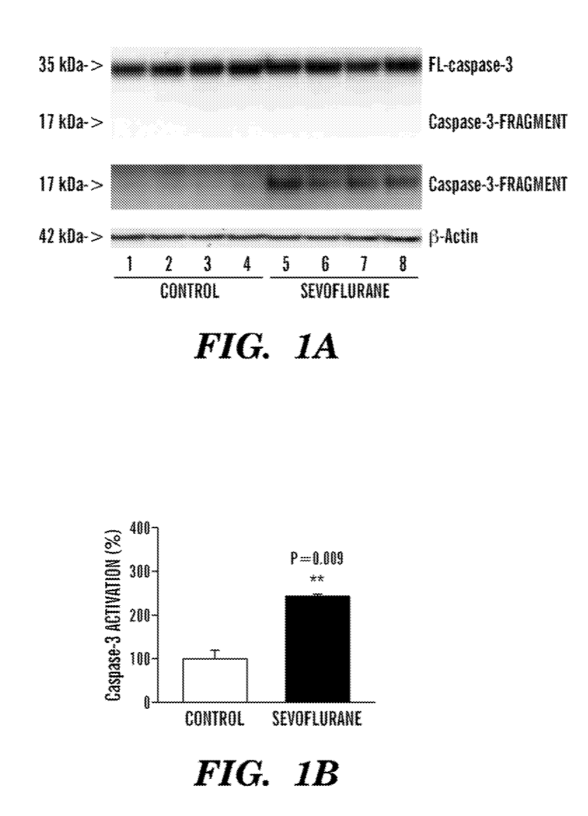

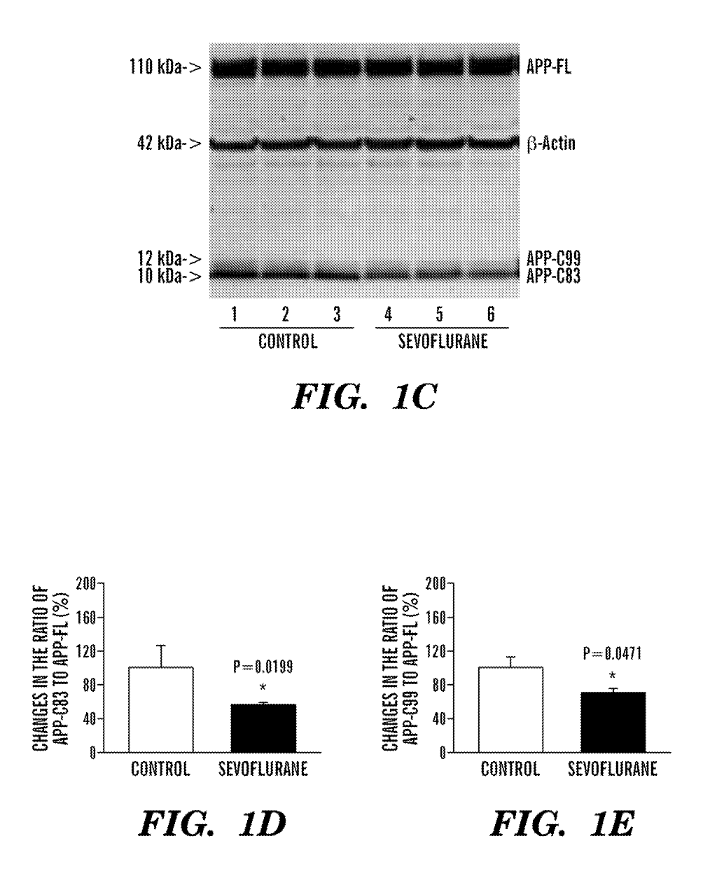

Caspase-3 activation is one of the final steps of cellular apoptosis (19). Therefore, the effects of sevoflurane on caspase-3 activation in the brain tissues of neonatal naïve mice were assessed by quantitative Western blot analyses. The 6-day-old neonatal naïve mice were treated with 3% sevoflurane plus 60% oxygen for 6 h, and the brain tissues were harvested at the end of the experiment and subjected to Western blot analysis by which caspase-3 antibody was used to detect both caspase-3 fragment (17-20 kDa) and full length (FL)-caspase-3 (35-40 kDa). FIG. 1A shows that sevoflurane anesthesia increases the protein levels of caspase-3 fragment in the brain tissues of neonatal naïve mice when compared with the control condition. The blot with only the caspase-3 fragment is the same Western blot with extended exposure time during the development of the film. Quantification of the Western blot, by determining the ratio of cleaved (activated) caspase-3 fragment (17-kDa) to FL-caspase-3 (35-40 kDa), revealed that sevoflurane anesthesia led to a 242% increase in caspase-3 cleavage (activation) when compared with the control condition (FIG. 1B; P=0.009).

Sevoflurane-induced caspase activation and apoptosis can lead to alterations in amyloid precursor protein (APP) processing in vitro (18). Accordingly, the effect of sevoflurane on APP processing in the brain tissues of neonatal mice was assessed herein. APP immunoblotting demonstrated visible decreases in the protein levels of APP-C83 and APP-C99 after the administration of anesthesia with 3% sevoflurane for 6 h when compared with control conditions (FIG. 1C). The quantification of the Western blot, by determining the ratio of APP-C-terminal fragments (APP-C83 fragment [10 kDa] and APP-C99 fragment [12 kDa]) to APP-FL (110 kDa), revealed that sevoflurane anesthesia led to a 45% and 33% decrease in the ratio of APP-C83 to APP-FL (FIG. 1D, P=0.0199) and APP-C99 to APP-FL (FIG. 1E, P=0.0471), respectively, when compared with the control condition in the brain tissues of neonatal naïve mice. These results indicate that sevoflurane can alter the APP processing by decreasing the levels of APP-C-terminal fragments (APP-C83 and APP-C99).

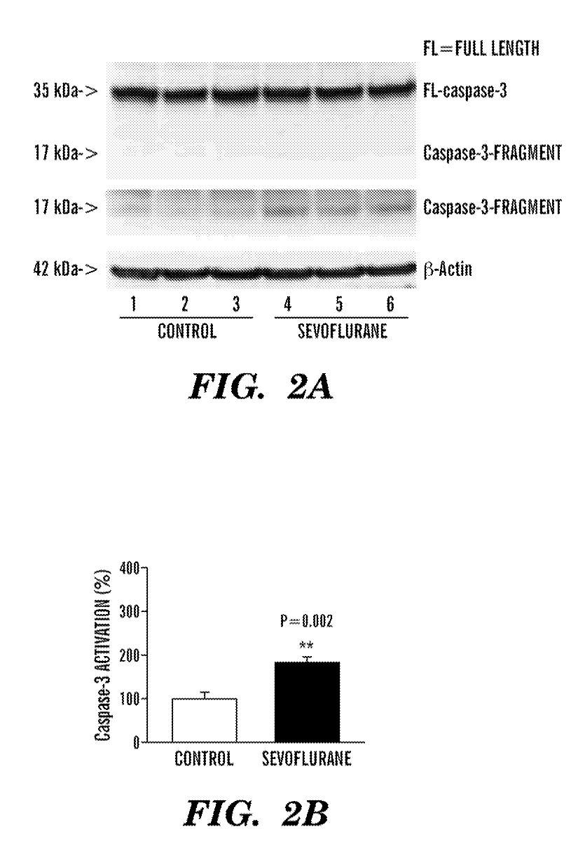

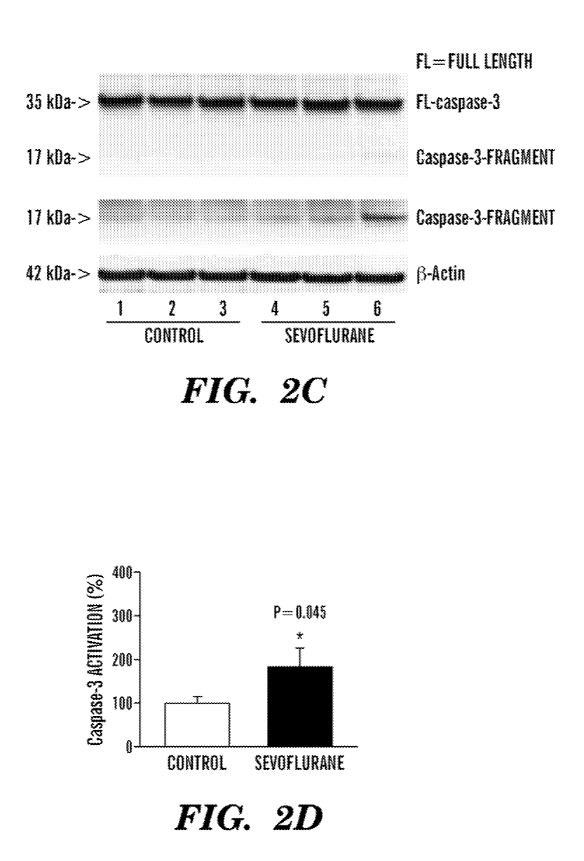

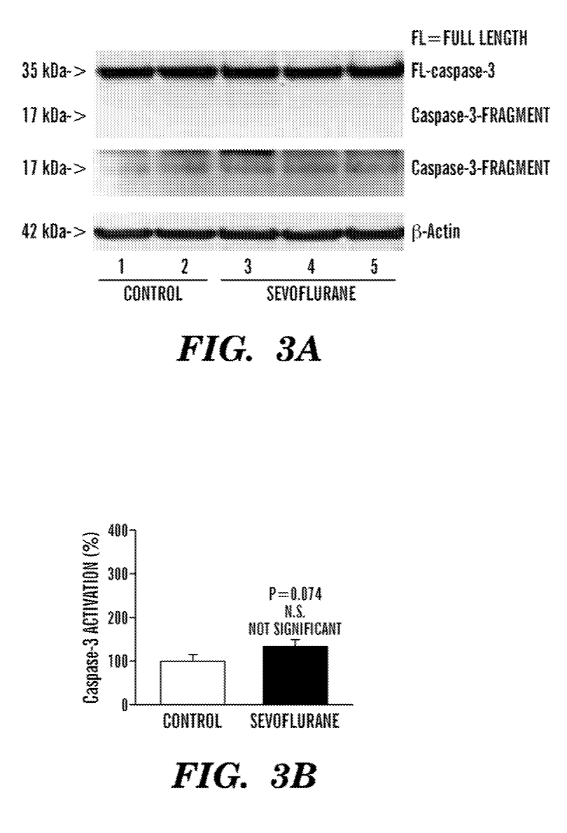

Next, the effect of a lower concentration of sevoflurane with the same treatment time (6 h) on caspase-3 activation in the brain tissues of neonatal mice was assessed. As demonstrated herein, anesthesia with 2.1% sevoflurane for 6 h induced caspase-3 activation in the brain tissues of neonatal naïve mice (FIGS. 2A and 2B): 100% versus 183%, P=0.002, and of neonatal AD transgenic mice (FIGS. 2C and 2D): 100% versus 178%, P=0.045. Further, the effect of anesthesia with the same concentration (3%) of sevoflurane but for a shorter treatment time on caspase-3 activation in the brain tissues of neonatal naïve mice was investigated. Anesthesia with 3% sevoflurane for 2 h did not increase caspase-3 activation (FIGS. 3A and 3B): 100% versus 128%, P=0.074. These results indicate that the commonly used inhalation anesthetic sevoflurane can induce caspase-3 activation in the brain tissues of neonatal mice in a time-dependent manner. This indicates that a specific length of treatment time (e.g., 6 h) of sevoflurane anesthesia can induce caspase activation in vivo.

### Example 3

### Sevoflurane Induced a Greater Degree of Caspase-3 Activation in Neonatal AD Transgenic Mice

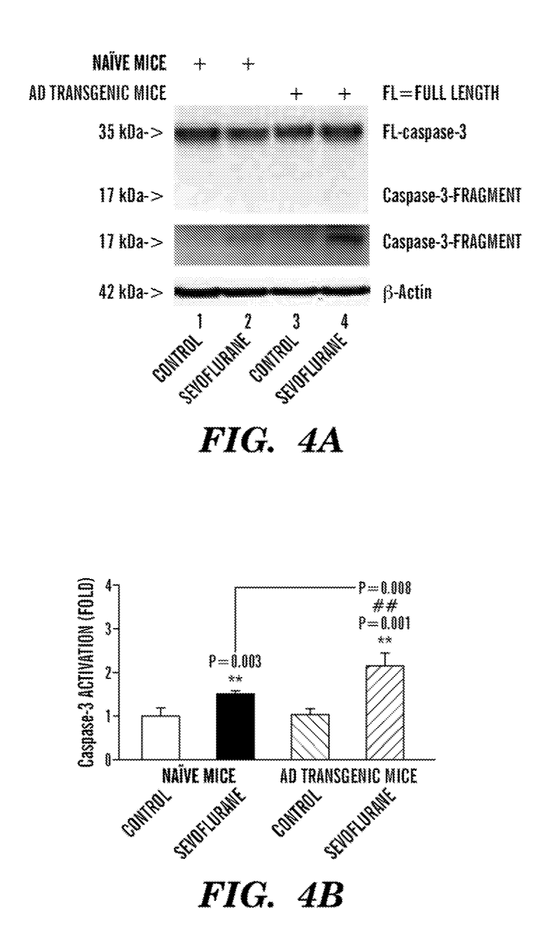

The effect of sevoflurane anesthesia on neurotoxicity in neonatal AD transgenic mice was assessed. The APPswe/PSEN1dE9 mouse is a particularly aggressive AD transgenic mouse model generated with mutant transgenes for APP (APPswe: KM594/5NL) and presenilin 1 (deletion of exon 9 [dE9]) (20). Using this APPswe/PSEN1dE9 mouse model, the effects of anesthesia with 3% sevoflurane for 6 h on caspase-3 activation in the brain tissues of 6-day-old AD transgenic mice were assessed and compared with that of 6-day-old naïve mice. FIG. 4A indicates that sevoflurane anesthesia induced visible increases in the protein levels of caspase-3 fragment, when compared with the control condition, in both neonatal naïve (lane 1 vs. lane 2) and AD transgenic mice (lane 3 vs. lane 4). The blot with only the caspase-3 fragment is the same Western blot with extended exposure time during the development of the film. Further, as demonstrated herein, sevoflurane anesthesia induced a more visible increase in the band of caspase-3 fragment in neonatal AD transgenic mice than that in neonatal naïve mice. Quantification of the Western blot demonstrated that sevoflurane anesthesia induced caspase-3 activation in the brain tissues of both neonatal naïve mice (1-fold vs. 1.48-fold, P=0.003) and AD transgenic mice (1-fold vs. 2.45-fold, P=0.001) (FIG. 4B). Moreover, sevoflurane anesthesia induced a greater degree of caspase-3 activation in the brain tissues of neonatal AD transgenic mice than that in neonatal naïve mice: 2.13-fold versus 1.48-fold, P=0.008 (FIG. 4B). (Onefold shown in FIG. 4B refers to the ratio of the activated (cleaved) caspase-3 fragment to FL-caspase-3 in the control group of either naïve mice or AD transgenic mice.) These results indicate that sevoflurane causes a greater degree of neurotoxicity in neonatal AD transgenic mice than in neonatal naïve mice.

### Example 4

### Sevoflurane Induced More TUNEL-Positive Cells in Neonatal AD Transgenic Mice

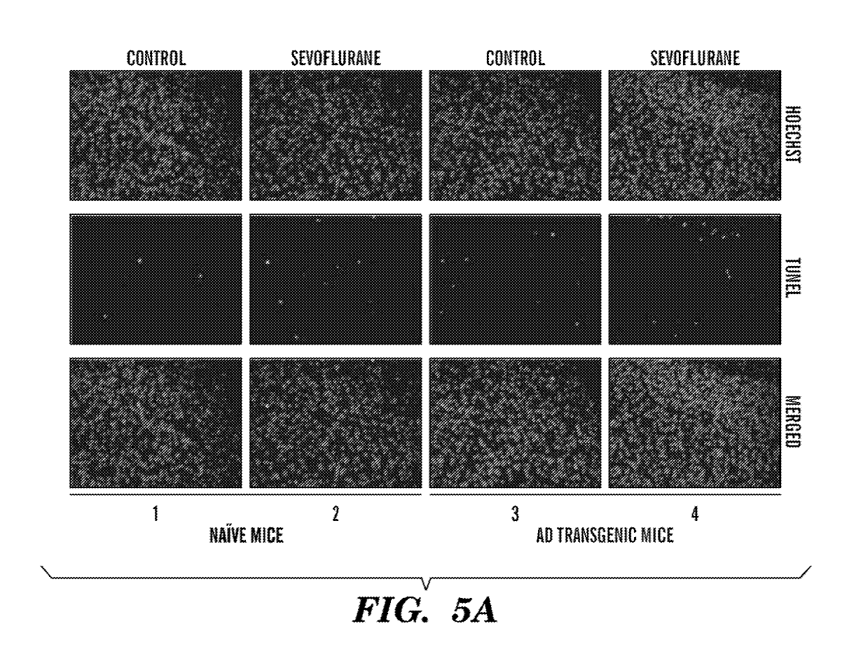

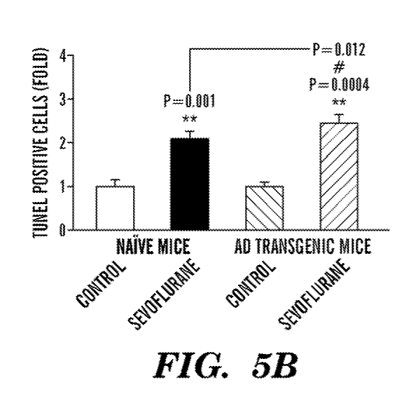

It has been previously discussed that caspase-3 activation alone may not represent apoptotic cell damage (21). Accordingly, the effects of 3% sevoflurane plus 60% oxygen for 6 h on cellular apoptosis were assessed using the TUNEL study. The TUNEL-positive cells were quantified using fold change. Onefold reported herein refers to the ratio of TUNEL-positive cells to the total cells in the control group of either naïve mice or AD transgenic mice. As demonstrated herein, sevoflurane anesthesia increased TUNEL-positive cells (apoptosis) when compared with the control condition in the brain tissues of neonatal naïve mice (FIGS. 5A and 5B; 1-fold vs. 2.08-fold; P=0.001) and neonatal AD transgenic mice (FIGS. 5A and 5B; 1-fold vs. 2.13-fold; P=0.0004). Consistent with the results of studies on caspase-3 activation described in FIG. 4, sevoflurane anesthesia induced more TUNEL-positive cells (apoptosis) in the brain tissues of neonatal AD transgenic mice than those in neonatal naïve mice: 2.45-fold versus 2.08-fold, P=0.012 (FIGS. 5A and 5B). Furthermore, double immunocytochemistry staining indicated that the majority of the TUNEL-positive cells in the brain tissues of neonatal AD transgenic mice after sevoflurane anesthesia were neurons (NeuN staining) but not microglia cells (Iba1 staining) or astrocytes (Glial fibrillary acidic protein staining) (FIG. 5C). These results indicate that sevoflurane can induce apoptosis in the neurons of the brain of neonatal mice, and that neonatal AD transgenic mice are more vulnerable to such sevoflurane-induced neurotoxicity.

### Example 5

### Sevoflurane Enhanced Aβ Levels in Neonatal Naive and AD Transgenic Mice

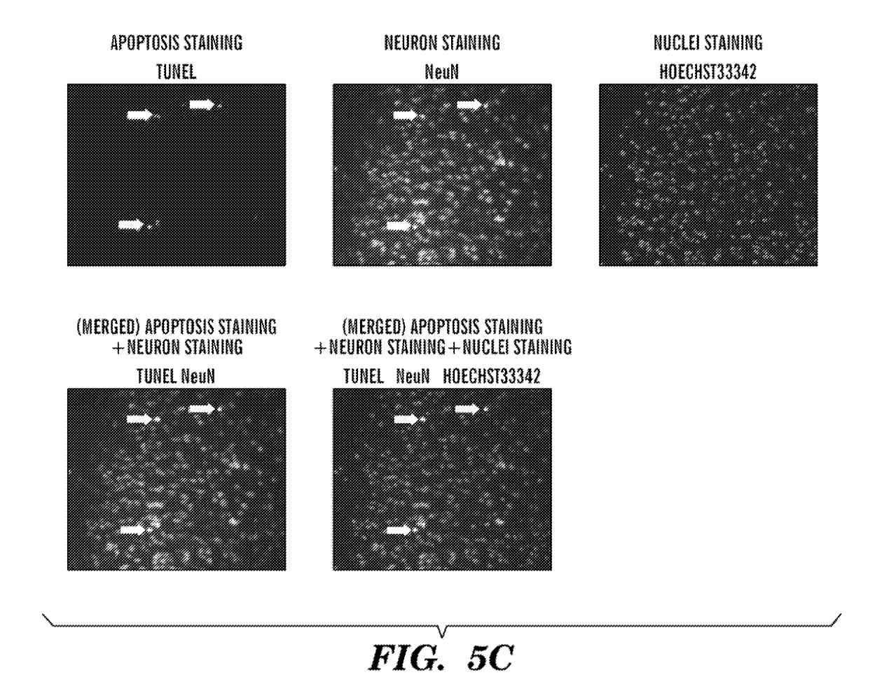

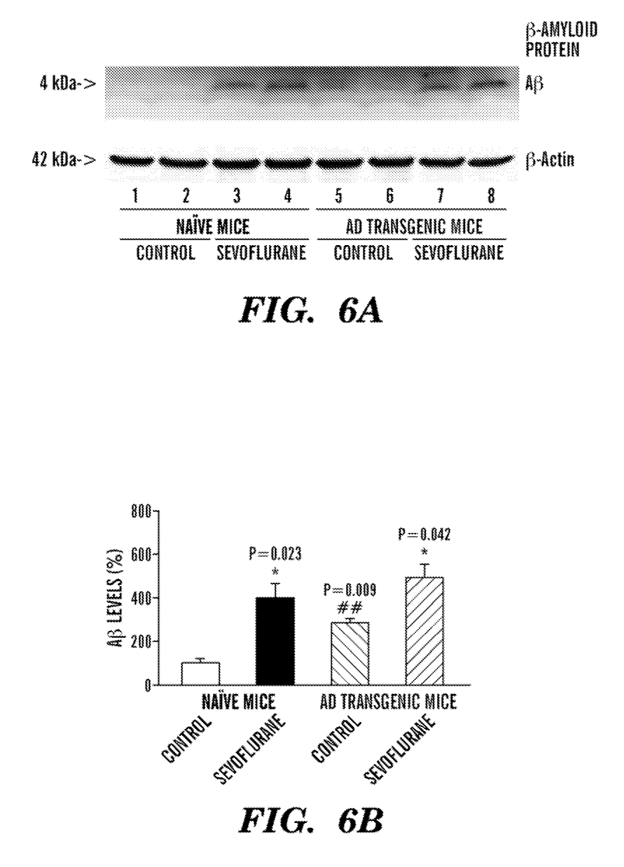

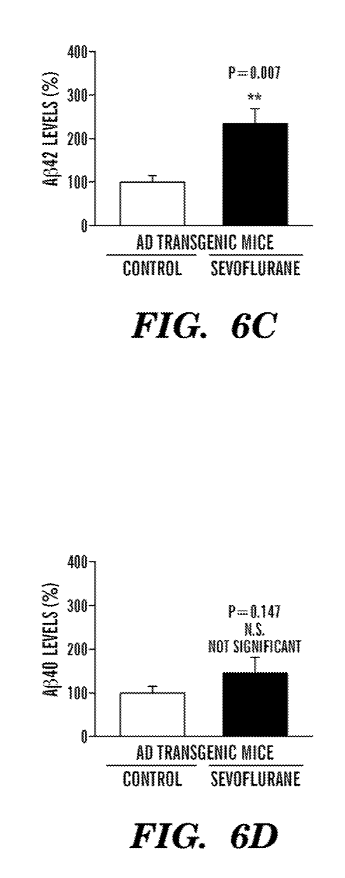

Sevoflurane has been shown previously to induce apoptosis, which then leads to Aβ accumulation in vitro and in the brain tissues of adult mice (18). Since sevoflurane can induce apoptosis and alter APP processing in the brain tissues of neonatal mice (FIGS. 1C to 1E), the effect of sevoflurane on enhancing Aβ levels in the brain tissues of these neonatal mice was assessed. The harvested brain tissues were subjected to Western blot analysis, by which antibody 6E10 was used to detect Aβ levels as described in Xie Z. et al (16). A13 immunoblotting revealed that an anesthesia administration of 3% sevoflurane for 6 h caused visible increases in Aβ levels in the Western blot when compared with control conditions (FIG. 6A). Quantification of the Western blot indicates that sevoflurane anesthesia increased Aβ levels in both neonatal naïve mice: 100% versus 401%, P=0.023, and AD transgenic mice: 287% versus 491%, P=0.042, when compared with the control condition (FIG. 6B), and that the baseline Aβ level in the brain tissues of neonatal AD transgenic mice was higher than those in neonatal naïve mice: 100% versus 287%, P=0.009. Furthermore, sandwich ELISA indicated that sevoflurane anesthesia increased Aβ42 levels: 100% versus 233%, P=0.007 (FIG. 6C), but not Aβ40 levels (FIG. 6D), in the brain tissues of neonatal AD transgenic mice, but not in the brain tissues of neonatal naïve mice. Anesthesia with 2.1% sevoflurane for 6 h increased the levels of Aβ42 in the brain tissues of neonatal AD transgenic mice (data not shown). These results indicate that sevoflurane specifically increases Aβ42 levels in the brain tissues of neonatal mice.

### Example 6

### IP3R Antagonist 2-aminoethoxydiphenyl borate (2-APB) Attenuated sevoflurane-Induced caspase-3 Activation and Increases in Aβ Levels

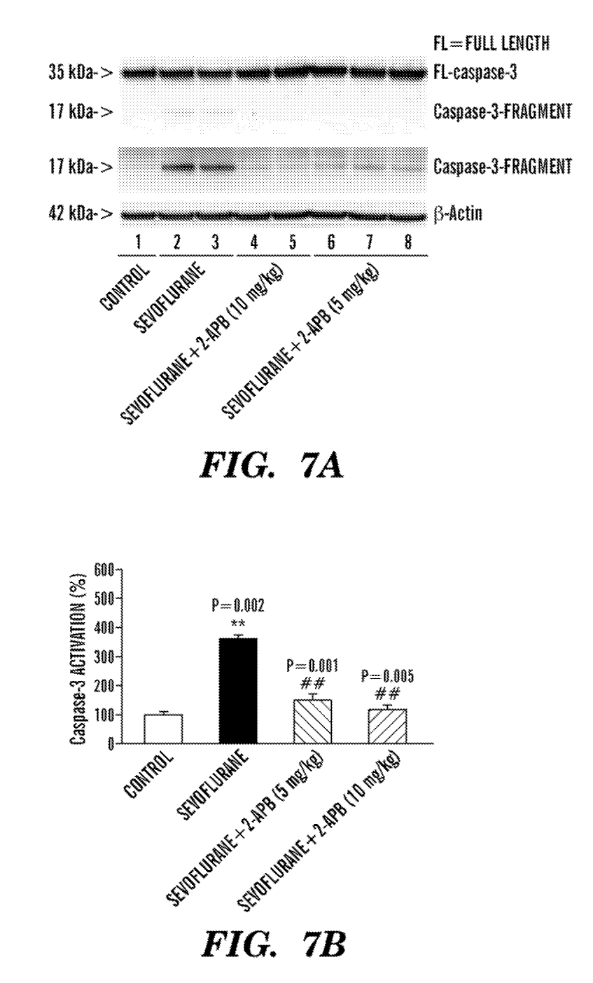

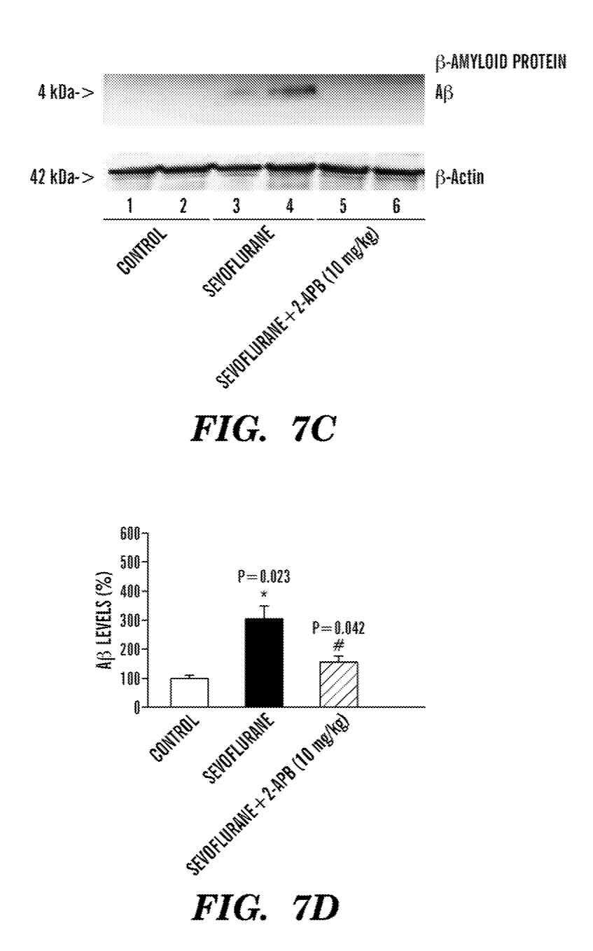

The underlying mechanism by which inhalation anesthetics induce apoptosis and enhance Aβ accumulation is largely unknown. Several studies have previously shown that the inhalation anesthetic isoflurane may increase cytosolic calcium levels, leading to apoptosis (22, 23). However, these previous studies did not teach or describe the use of a specific IP3R antagonist, particularly, 2APB as disclosed herein, for inhibition of caspase-3 activation in brain cells of an in vivo mouse model or for prevention of POCD as disclosed herein. The effects of the IP3R antagonist 2-APB on sevoflurane-induced caspase-3 activation and Aβ accumulation in the brain tissues of neonatal naïve mice were assessed. Anesthesia with 3% sevoflurane for 6 h led to caspase-3 activation when compared with the control condition, but five milligram per kilogram (FIG. 7A: lanes 6-8) and 10 mg/kg (FIG. 7A: lanes 4 and 5) of 2-APB attenuated sevoflurane-induced caspase-3 activation in a dose-dependent manner. Quantification of the Western blot indicated that sevoflurane anesthesia induced caspase-3 activation: 100% versus 356%, P=0.002, and the IP3R antagonist 2-APB attenuated sevoflurane-induced caspase-3 activation in a dose-dependent manner, 5 mg/kg 2-APB (gray bar): 356% versus 149%, P=0.001; 10 mg/kg 2-APB (hatched bar): 356% versus 115%, P=0.005 (FIG. 7B). 2-APB also attenuated sevoflurane-induced increases in Aβ levels (FIGS. 7C and 7D), 304% versus 157%, P=0.042. These results indicate that IP3R is involved in sevoflurane-induced caspase activation, apoptosis, and Aβ accumulation.

### Example 7

### Sevoflurane Increased TNF-α Levels in Neonatal AD Transgenic Mice

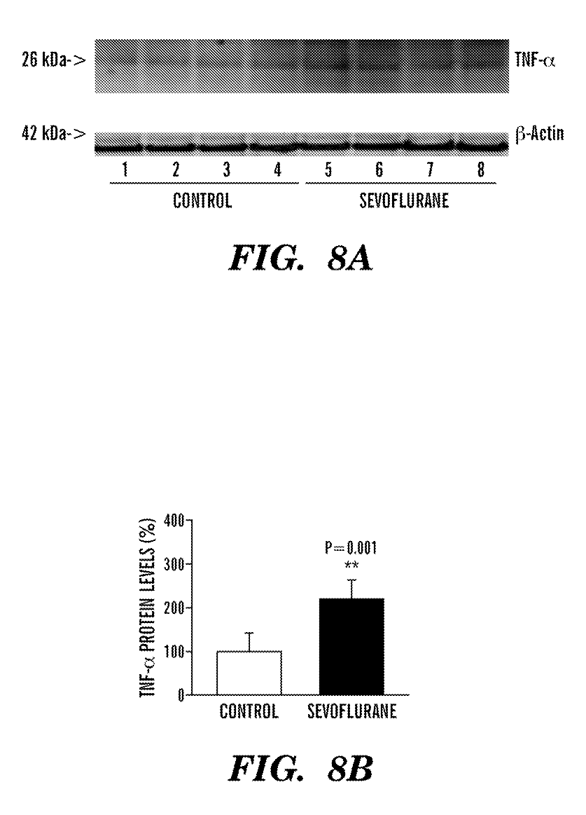

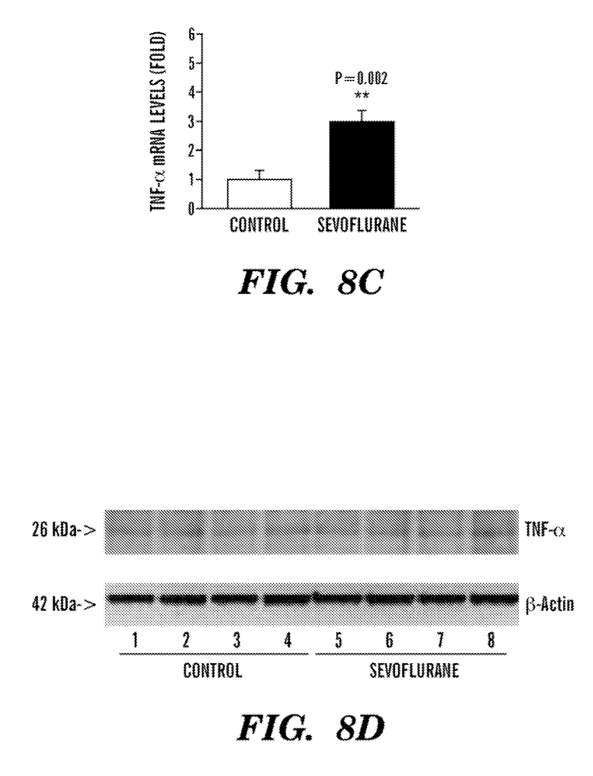

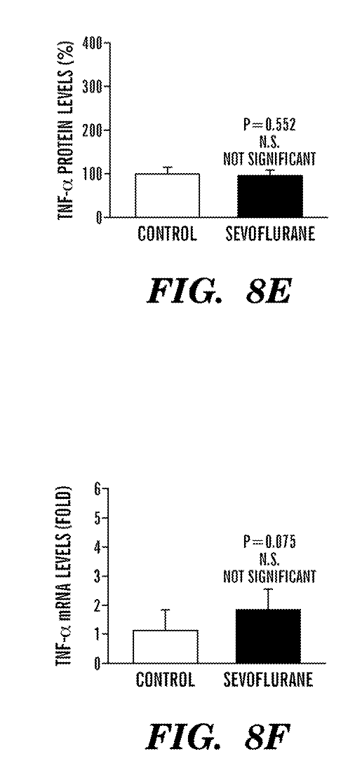

Previous studies have suggested that neurons and microglia cells can produce inflammatory mediators including the proinflammatory cytokine TNF-α (24). TNF-α is a death-inducing cytokine, which can induce both apoptosis and necrosis through receptor-interacting protein 3, a protein kinase (25). Accordingly, the effects of sevoflurane on neuroinflammation were assessed by determining TNF-α levels in the brain tissues of neonatal naïve and AD transgenic mice after administration of 3% sevoflurane anesthesia for 6 h. Sevoflurane anesthesia increased protein levels (FIGS. 8A and 8B, 100% vs. 219%, P=0.001) and messenger RNA levels (FIG. 8C, P=0.002) of TNF-α levels in the brain tissues of neonatal AD transgenic mice but not in the brain tissues of neonatal naïve mice (FIGS. 8D to 8F). These results indicate that sevoflurane increases TNF-α levels by enhancing its generation in the brain tissues of neonatal AD transgenic mice, leading to neuroinflammation.

Several previous studies have suggested that anesthesia may be a significant risk factor in children for the later development of learning disabilities and/or deviant behavior (1, 2). However, these studies do not demonstrate whether anesthesia can contribute to the development of the learning disability and/or deviant behavior. It is possible that the need for anesthesia is a marker for unidentified factors, rather than anesthesia itself, that contribute to the development of the hearing disability and/or deviant behavior. Thus, it was important to assess the effects of sevoflurane, the most commonly used inhalation anesthetic (especially in pediatric patients), on the biochemical changes that are associated with cognitive dysfunction in neonatal mice, which include apoptosis, Aβ accumulation, and neuroinflammation, as disclosed in the Examples.

As described herein, anesthesia with 3% or 2.1% sevoflurane for 6 h, but not 3% sevoflurane for 2 h, induced caspase activation and apoptosis, altered APP processing, and increased Aβ levels in the brain tissues of neonatal naïve and AD transgenic mice. These results indicated that sevoflurane induces caspase activation and apoptosis in a time-dependent manner, and sevoflurane anesthesia specifically induces apoptosis in neurons and increase Aβ42 levels. Moreover, sevoflurane anesthesia induced a greater degree of caspase activation and apoptosis in the brain tissues of neonatal AD transgenic mice (B6.Cg-Tg[APPswe, PSEN1dE9]85 Dbo/J) than in neonatal naïve mice. Additionally, sevoflurane anesthesia induced neuroinflammation by increasing proinflammatory cytokine TNF-α in the brain tissues of AD transgenic mice, but less likely in the brain tissues of naïve mice. Collectively, these results indicate that sevoflurane anesthesia leads to neurotoxicity by inducing apoptosis and neuroinflammation and by increasing Aβ levels in the brain tissues of neonatal mice, and the overexpression of AD genes and/or increased Aβ levels in AD transgenic mice potentiates such neurotoxicity. These findings indicate that general anesthetics, such as sevoflurane, the mostly commonly used inhalation anesthetic, increases Aβ accumulation in the brain of individuals with increased Aβ burden. Such patients include patients with Down syndrome, the unaffected carriers of APP or presenilin gene mutations, and the late onset AD risk factor, apolipoprotein e-E4.

While previous in vitro studies discuss the alteration of amyloid precursor protein (APP) processing after sevoflurane anesthesia (18), the results presented herein show, for the first time, in an AD transgenic mouse in vivo model that sevoflurane anesthesia can reduce the levels of APP-C-terminal fragments including APP-C83 and APP-C99. Without wishing to be bound by theory, since APP-C83 and APP-C99 are metabolized by 8-secretase (reviewed in 10, 12), sevoflurane may increase the activity of 8-secretase, leading to reductions in the levels of APP-C83 and APP-C99. Sevoflurane and other anesthetics may also influence the levels of 8-secretase components, for example, presenilin 1, nicastrin, presenilin enhancer 2, and anterior pituitary hormones (26-30), and the 8-secretase activity (31).

Sevoflurane anesthesia induced neuroinflammation in the brain tissues of AD transgenic mice far more than in the brain tissues of neonatal naïve mice. Both A3 accumulation and neuroinflammation are important parts of AD neuropathogenesis, and they can potentiate each other's neurotoxicity (32, 33; reviewed in 11). It is envisioned that the higher baseline levels of Aβ in AD transgenic mice facilitates the effects of sevoflurane on increasing TNF-α levels, leading to apparent neuroinflammation. While sevoflurane induces caspase activation in a time-dependent manner (FIG. 3), it is likely that sevoflurane also produces dose- and time-dependent effects on the levels of TNF-α and other proinflammation cytokines (e.g., interleukin-6) in both naïve and AD transgenic mice.

Even though baseline A levels in the brain of B6.Cg-Tg (APPswe, PSEN1dE9)85Dbo/J mice were higher than those in the brain of the naïve mice, sevoflurane anesthesia did not lead to significantly greater increases of Aβ levels in the brain tissues of AD transgenic mice than in naïve mice. This could be because of the ceiling effects of sevoflurane-induced increases in Aβ levels. It is also possible that sevoflurane can enhance Aβ levels through a nonapoptosis pathway. A recent study (34) has shown that cellular stress induced by glucose deprivation can lead to increases in the levels of 3-secretase (the enzyme to generate Aβ) and Aβ through phosphorylation of the translation initiation factor eIF2α independent of caspase activation and apoptosis. It is envisioned that anesthetics also increase Aβ generation through this translation mechanism, e.g., phosphorylation of the translation initiation factor eIF2α.

Nevertheless, ELISA studies showed that sevoflurane anesthesia enhanced the levels of Aβ42, but not Aβ40, in the brain tissues of neonatal AD transgenic mice, but less likely in the brain tissues of neonatal naïve mice. It is possible that the ELISA kit used in these experiments was not sensitive enough to detect the non-human Aβ levels in the brain tissues of neonatal naïve mice. Nevertheless, this result indicates that sevoflurane anesthesia can lead to a greater degree of Aβ accumulation in the brain tissues of AD transgenic mice than that in naïve mice.

The IP3 receptor, located in the endoplasmic reticulum membrane, regulates the release of calcium from the endoplasmic reticulum to the cytoplasm (35 reviewed in 36). The results presented herein indicate that the IP3R antagonist 2-APB (37) attenuates sevoflurane-induced caspase-3 activation and Aβ accumulation in neonatal naïve mice. This indicates that sevoflurane acts on IP3R to affect calcium homeostasis, leading to apoptosis and Aβ accumulation. Moreover, this indicates that 2-APB prevents or reduces sevoflurane-induced neurotoxicity. Further, it is expected that IP3 antagonism leads to many other effects. It is envisioned that other IP3 antagonists, for example, xestospongin C, attenuate sevoflurane-induced caspase activation and Aβ accumulation in brain cells.

Although blood gas was not measured in each of the mice after the administration of anesthesia with 3% sevoflurane plus 60% oxygen for 6 h, the same sevoflurane anesthesia has been shown not to significantly alter blood gas and brain blood flow (6), which is consistent with the pilot studies performed (Table 3). In addition, the result that anesthesia with 3% sevoflurane plus 60% oxygen for 2 h does not induce casase-3 activation further indicates that it is sevoflurane, but not physiologic changes (e.g., alterations in oxygen, carbon dioxide or pH in blood), that causes neurotoxicity. It is also possible that the combination of sevoflurane and anesthesia-induced hypoxia and/or acidosis induced neurotoxicity in some mice in the experiments disclosed herein.

TABLE 3
Blood gas of neonatal mice under control condition and sevoflurane
anesthesia

| | | 3% Sevoflurane + |
|---|---|---|
| | 60% Oxygen | 60% Oxygen |
| pH | 7.33 ± 0.05 | 7.32 ± 0.06 |
| PaO2 | 174 ± 12.4 mmHg | 152 ± 29.8 mmHg |
| PaCO2 | 48 ± 5.1 mmHg | 52 ± 7.8 mmHg |

In conclusion, the results presented herein show that sevoflurane, the most commonly used inhalation anesthetic, induces caspase activation and apoptosis, alters APP processing, and increases Aβ levels in the brain tissues of neonatal naïve and AD transgenic mice. Importantly, more severe apoptosis, Aβ accumulation, and neuroinflammation occurred in the brain tissues of neonatal AD transgenic mice when compared with neonatal naïve mice. These results indicate that sevoflurane may cause neurotoxicity in neonatal mice and that overexpression of mutated AD genes, that is, presenilin 1 and APP, and/or increased A3 levels in AD transgenic mice may potentiate such neurotoxicity. Further, sevoflurane-induced neurotoxicity was associated with IP3R, and 2-APB, one of the IP3R antagonists, attenuates sevoflurane-induced neurotoxicity. Given the findings that anesthesia could be a risk factor for the development of a learning disability in children and that sevoflurane is used extensively in pediatric patients, these findings should lead to the prevention of anesthesia-induced learning disabilities in children.

### Example 8

### Effects of Propofol on Isoflurane-Induced Caspase Activation and Aβ Oligomerization

Alzheimer's disease (AD) is the most common form of age-related dementia and one of the most serious health problems in the U.S. The accumulation and oligomerization of β-amyloid protein (Aβ) is a key pathological event in AD. Previous studies suggest that caspase activation and apoptosis are associated with a variety of neurodegenerative disorders, including AD (12). The commonly used inhalation anesthetic isoflurane has been previously reported to enhance Aβ oligomerization and cytotoxicity (38), as well as to induce caspase activation and apoptosis, and to increase Aβ generation (39). Further, isoflurane may induce caspase activation and apoptosis through isoflurane-induced Aβ oligomerization (40). Intravenous anesthetic propofol has been suggested to decrease A3 oligomerization in vitro (38). As such, the following experiments were performed to determine if propofol can attenuate the caspase activation induced by inhalational anesthetics (e.g., isoflurane) by decreasing Aβ oligomerization.

To this end, H4 human neuroglioma cells stably transfected to express human full-length wild-type APP (H4-APP cells) in cell culture media with 7.5 M Aβ40, Aβ42 or Aβ40 plus Aβ42, were treated with an clinically relevant concentration of propofol (e.g., 100 μM) for one hour, and then with 2% isoflurane (plus 21% O2 and 5% CO2) for six hours. Wild-type H4 human neuroglioma cells (H4 naïve cells) and primary neurons from naïve (C57BL/6) mice were also used in the study for comparison. To obtain primary neurons, naïve (C57BL/6) mice with gestation stage of day 15 were euthanized with CO₂. The primary neurons were harvested from the embyros. The harvested neurons were cultured for 7 to 10 days prior to treatment.

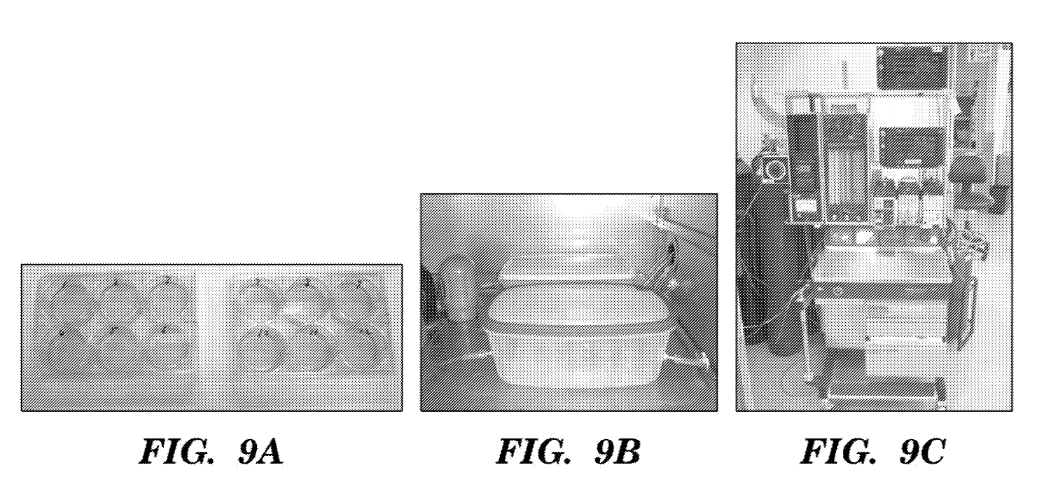

Anesthesia machine was used for the delivery of 2% isoflurane to the cells or neurons plated in a six well plate in a sealed plastic box located in a 37° C. incubator and Datex infrared gas analyzer was used for the monitoring (FIGS. 9A to 9C). The cells and the cell culture media were harvested at the end of the treatment. Caspase-3 activation and Aβ oligomerization were measured with quantitative Western blotting. Chemiluminescence signal from the western blot membranes was captured using a VersaDoc Imaging System and images were analyzed using Quantity One software (BioRad Hercules, Calif.).

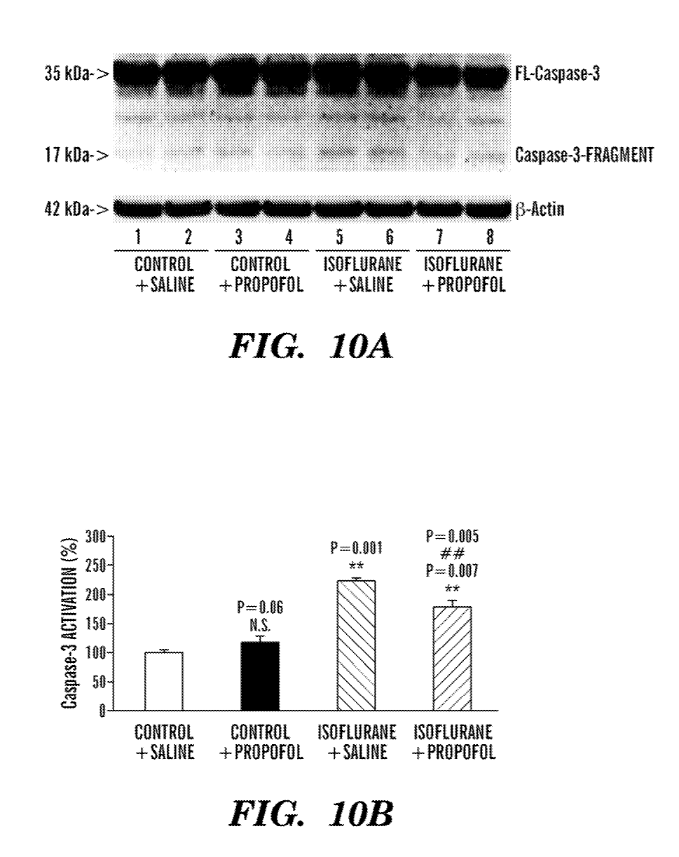

Apoptosis is a programmed cell death requiring cysteine proteases called caspases, and caspase-3 activation is one of the final steps of apoptosis. Thus, a caspase-3 antibody (Cell Signaling, Danvers, Mass.) was used to recognize full-length (35 kDa), and the large fragment of cleaved caspase-3 (17-20 kDa). As demonstrated herein, anesthesia with 2% isoflurane led to caspase-3 activation in H4-APP cells, when compared with the control condition with saline administered. Treatment of H4-APP cells with 100 μM propofol (a GABA receptor agonist) for one hour prior to isoflurane anesthesia attenuated isoflurane-induced caspase-3 activation in H4-APP cells (FIGS. 10A and 10B).

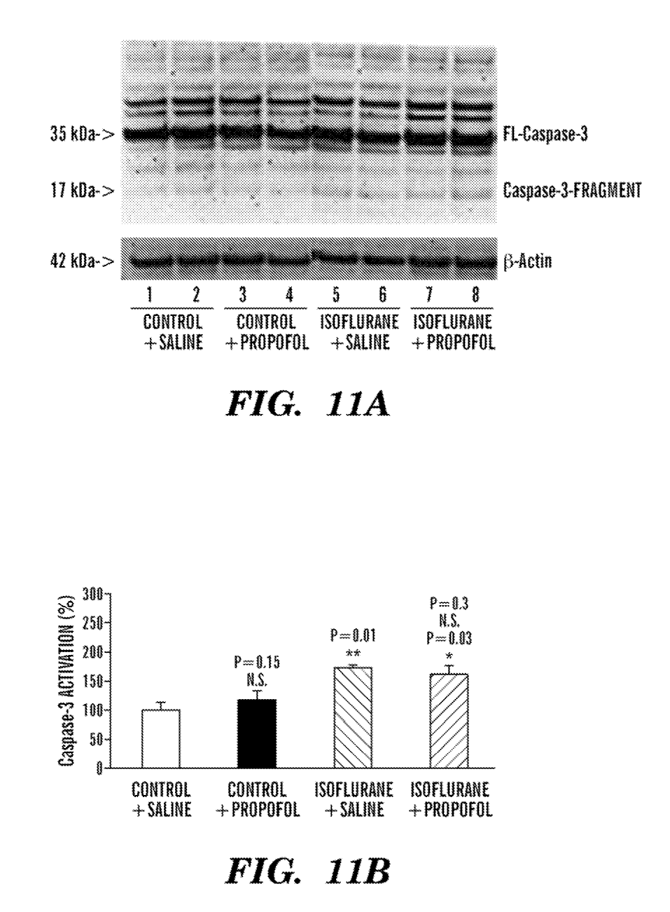

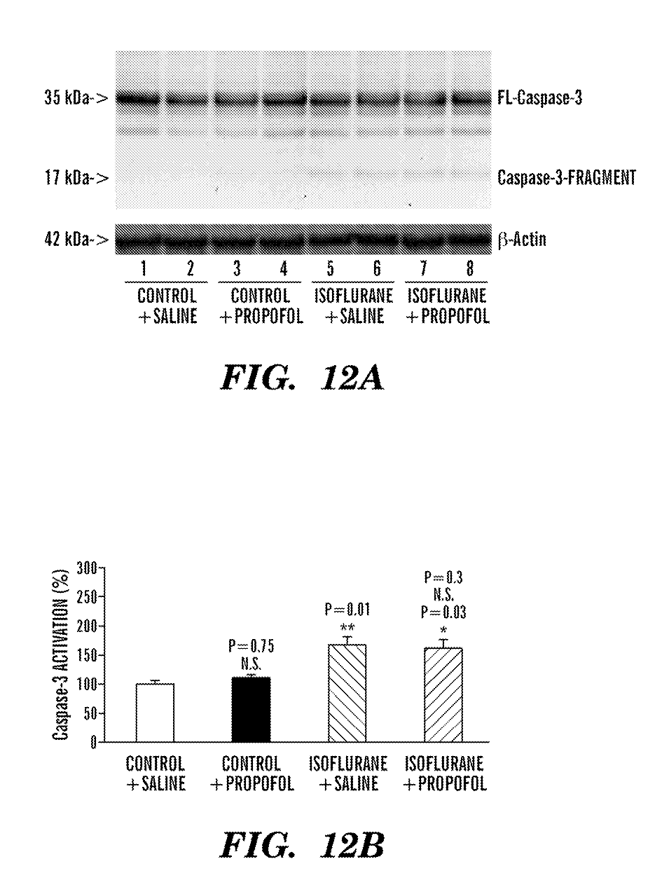

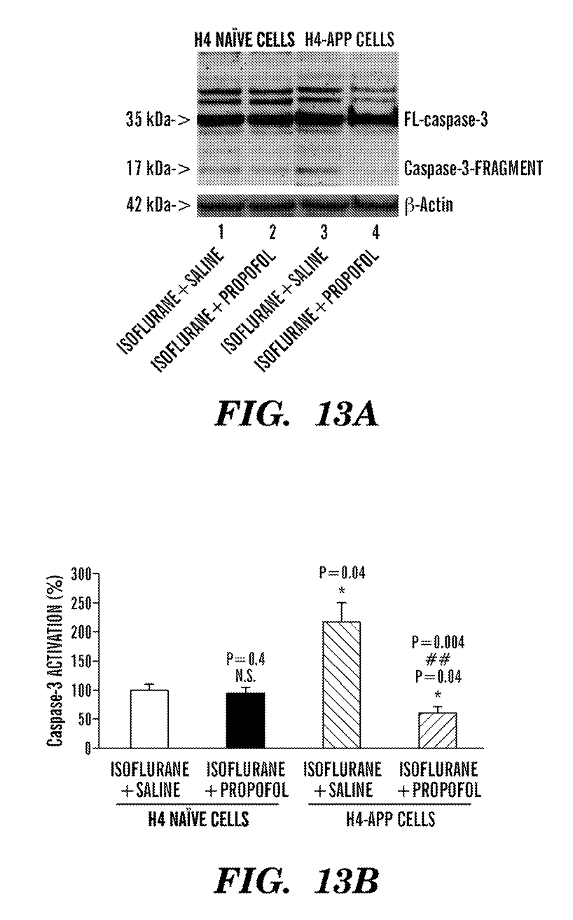

Next, H4 naïve cells were used to assess the effects of propofol on isoflurane-induced caspase activation. Propofol treatment did not attenuate the isoflurane-induced caspase-3 activation in H4 naïve cells (FIGS. 11A and 11B). Further, it was demonstrated that propofol did not attenuate the isoflurane-induced caspase-3 activation in primary neurons from the naïve (C57BL/6) mice with gestation stage of day 15 (FIGS. 12A and 12B). FIGS. 13A and 13B show the comparison of the propofol effects on isoflurane-induced caspase 3-activation between the H4-APP and H4 naïve cells.

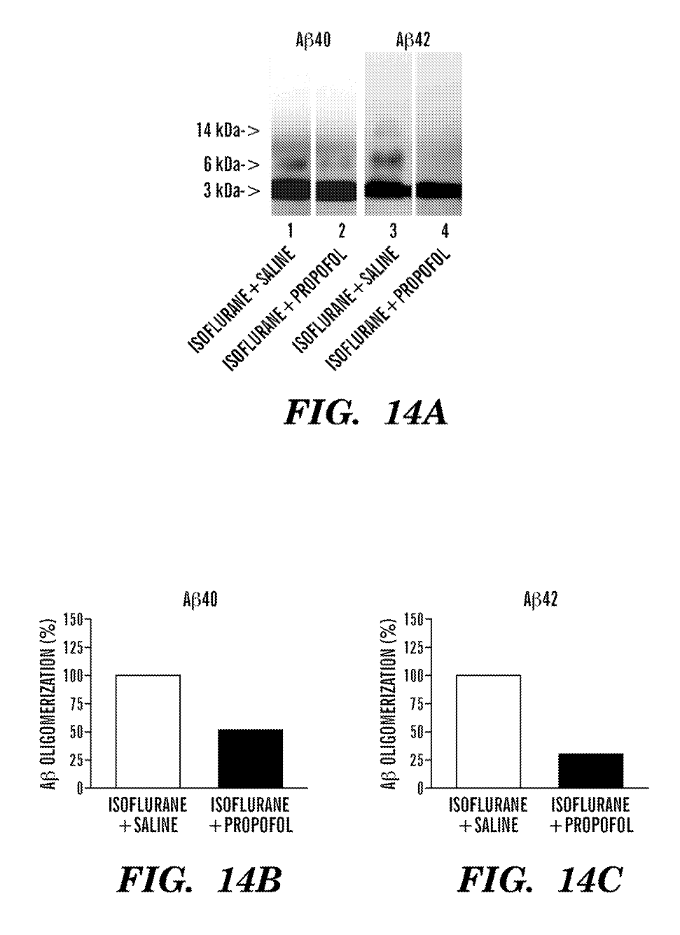

In addition to the propofol effects on caspase-3 activation, its effects on Aβ oligomerization were assessed. H4-APP cell culture media were exposed to 2% isoflurane with and without 100 μM propofol for six hours. Antibody mAb 6E10 (1:3,000, Sigma) was used to detect Aβ40 and Aβ42 oligomerization. As shown herein, treatment of H4-APP cells with 100 μM propofol for one hour prior to isoflurane anesthesia attenuated isoflurane-induced Aβ40 and Aβ42 in H4-APP cells (FIGS. 14A to 14C).

In conclusion, propofol alone did not induce caspase-3 activation or Aβ oligomerization. However, propofol attenuated the isoflurane-induced caspase-3 activation in H4-APP cells, but not in H4 naïve cells. Further, propofol decreased the isoflurane-caused Aβ oligomerization. Propofol attenuated the isoflurane-induced caspase activation and without wishing to be bound by theory, this is thought to occur by decreasing the Aβ oligomerization. These results indicate that propofol can be used to treat the anesthesia-induced neurotoxicity.

### REFERENCES

1. Wilder R T, Flick R P, Sprung J, Katusic S K, Barbaresi W J, Mickelson C, Gleich S J, Schroeder D R, Weaver A L, Warner D O: Early exposure to anesthesia and learning disabilities in a population-based birth cohort. ANESTHESIOLOGY 2009; 110:796-804
2. Kalkman C J, Peelen L, Moons K G, Veenhuizen M, Bruens M, Sinnema G, de Jong T P: Behavior and development in children and age at the time of first anesthetic exposure. ANESTHESIOLOGY 2009; 110:805-12
3. Bartels M, Althoff R R, Boomsma D I: Anesthesia and cognitive performance in children: No evidence for a causal relationship. Twin Res Hum Genet. 2009; 12:246-53
4. Stratmann G, May L D, Sall J W, Alvi R S, Bell J S, Ormerod B K, Rau V, Hilton J F, Dai R, Lee M T, Visrodia K H, Ku B, Zusmer E J, Guggenheim J, Firouzian A: Effect of hypercarbia and isoflurane on brain cell death and neurocognitive dysfunction in 7-day-old rats. ANESTHESIOLOGY 2009; 110:849-61
5. Stratmann G, Sall J W, May L D, Bell J S, Magnusson K R, Rau V, Visrodia K H, Alvi R S, Ku B, Lee M T, Dai R: Isoflurane differentially affects neurogenesis and long-term neurocognitive function in 60 day-old and 7-day-old rats. ANESTHESIOLOGY 2009; 110:834-48
6. Satomoto M, Satoh Y, Terui K, Miyao H, Takishima K, Ito M, Imaki J: Neonatal exposure to sevoflurane induces abnormal social behaviors and deficits in fear conditioning in mice. ANESTHESIOLOGY 2009; 110:628-37
7. Goate A, Chartier-Harlin M C, Mullan M, Brown J, Crawford F, Fidani L, Giuffra L, Haynes A, Irving N, James L, Mant R, Newton P, Rooke K, Roques P, Talbot C, Pericak-Vance M, Roses A, Williamson R, Rossor M, Owen M, Hardy J: Segregation of a missense mutation in the amyloid precursor protein gene with familial Alzheimer's disease. Nature 1991; 349:704-6
8. Masters C L, Simms G, Weinman N A, Multhaup G, McDonald B L, Beyreuther K: Amyloid plaque core protein in Alzheimer disease and Down syndrome. Proc Natl Acad Sci USA 1985; 82:4245-9
9. Selkoe D J, Podlisny M B, Joachim C L, Vickers E A, Lee G, Fritz L C, Oltersdorf T: Beta-amyloid precursor protein of Alzheimer disease occurs as 110- to 135-kilodalton membrane-associated proteins in neural and normeural tissues. Proc Natl Acad Sci USA 1988; 85:7341-5
10. Tanzi R E, Bertram L: Alzheimer's disease: The latest suspect. Nature 2008; 454:706-8
11. Selkoe D J: Alzheimer's disease: Genes, proteins, and therapy. Physiol Rev 2001; 81:741-66
12. Xie Z, Tanzi R E: Alzheimer's disease and post-operative cognitive dysfunction. Exp Gerontol 2006; 41:346-59
13. Tan Z S, Beiser A S, Vasan R S, Roubenoff R, Dinarello C A, Harris T B, Benjamin E J, Au R, Kiel D P, Wolf P A, Seshadri S: Inflammatory markers and the risk of Alzheimer disease: The Framingham Study. Neurology 2007; 68:1902-8
14. Wyss-Coray T: Inflammation in Alzheimer disease: Driving force, bystander or beneficial response? Nat Med 2006; 12:1005-15
15. Heneka M T, O'Banion M K: Inflammatory processes in Alzheimer's disease. J Neuroimmunol 2007; 184:69-91
16. Xie Z, Culley D J, Dong Y, Zhang G, Zhang B, Moir R D, Frosch M P, Crosby G, Tanzi R E: The common inhalation anesthetic isoflurane induces caspase activation and increases amyloid beta-protein level in vivo. Ann Neurol 2008; 64:618-27
17. Fukuchi K, Pham D, Hart M, Li L, Lindsey J R: Amyloid-beta deposition in skeletal muscle of transgenic mice: Possible model of inclusion body myopathy. Am J Pathol 1998; 153:1687-93
18. Dong Y, Zhang G, Zhang B, Moir R D, Xia W, Marcantonio E R, Culley D J, Crosby G, Tanzi R E, Xie Z: The common inhalational anesthetic sevoflurane induces apoptosis and increases beta-amyloid protein levels. Arch Neurol 2009; 66:620-31
19. Thornberry N A, Lazebnik Y: Caspases: Enemies within. Science 1998; 281:1312-6
20. Garcia-Alloza M, Robbins E M, Zhang-Nunes S X, Purcell S M, Betensky R A, Raju S, Prada C, Greenberg S M, Bacskai B J, Frosch M P: Characterization of amyloid deposition in the APPswe/PS1dE9 mouse model of Alzheimer disease. Neurobiol Dis 2006; 24:516-24
21. McLaughlin B, Hartnett K A, Erhardt J A, Legos J J, White R F, Barone F C, Aizenman E: Caspase 3 activation is essential for neuroprotection in preconditioning. Proc Natl Acad Sci USA 2003 100:715-20
22. Zhang G, Dong Y, Zhang B, Ichinose F, Wu X, Culley D J, Crosby G, Tanzi R E, Xie Z: Isoflurane-induced caspase-3 activation is dependent on cytosolic calcium and can be attenuated by memantine. J Neurosci 2008; 28:4551-60
23. Wei H, Liang G, Yang H, Wang Q, Hawkins B, Madesh M, Wang S, Eckenhoff R G: The common inhalational anesthetic isoflurane induces apoptosis via activation of inositol 1,4,5-trisphosphate receptors. ANESTHESIOLOGY 2008; 108:251-60
24. Gong C, Qin Z, Betz A L, Liu X H, Yang G Y: Cellular localization of tumor necrosis factor alpha following focal cerebral ischemia in mice. Brain Res 1998; 801:1-8
25. He S, Wang L, Miao L, Wang T, Du F, Zhao L, Wang X: Receptor interacting protein kinase-3 determines cellular necrotic response to TNF-alpha. Cell 2009; 137:1100-11
26. Li Y M, Lai M T, Xu M, Huang Q, DiMuzio-Mower J, Sardana M K, Shi X P, Yin K C, Shafer J A, Gardell S J: Presenilin 1 is linked with gamma-secretase activity in the detergent solubilized state. Proc Natl Acad Sci USA 2000; 97:6138-43
27. Yu G, Nishimura M, Arawaka S, Levitan D, Zhang L, Tandon A, Song Y Q, Rogaeva E, Chen F, Kawarai T, Supala A, Levesque L, Yu H, Yang D S, Holmes E, Milman P, Liang Y, Zhang D M, Xu D H, Sato C, Rogaev E, Smith M, Janus C, Zhang Y, Aebersold R, Farrer L S, Sorbi S, Bruni A, Fraser P, St George-Hyslop P: Nicastrin modulates presenilin-mediated notch/glp-1 signal transduction and betaAPP processing. Nature 2000; 407:48-54
28. Francis R, McGrath G, Zhang J, Ruddy D A, Sym M, Apfeld J, Nicoll M, Maxwell M, Hai B, Ellis M C, Parks A L, Xu W, Li J, Gurney M, Myers R L, Himes C S, Hiebsch R, Ruble C, Nye J S, Curtis D: Aph-1 and pen-2 are required for notch pathway signaling, gamma-secretase cleavage of betaAPP, and presenilin protein accumulation. Dev Cell 2002; 3:85-97
29. Steiner H, Winkler E, Edbauer D, Prokop S, Basset G, Yamasaki A, Kostka M, Haass C: PEN-2 is an integral component of the gamma-secretase complex required for coordinated expression of presenilin and nicastrin. J Biol Chem 2002; 277:39062-5
30. Xie Z, Romano D M, Tanzi R E: Effects of RNAi-mediated silencing of PEN-2, APH-1a, and nicastrin on wild-type vs FAD mutant forms of presenilin 1. J Mol Neurosci 2005; 25:67-77
31. Cao X, Sudhof T C: A transcriptionally [correction of transcriptively] active complex of APP with Fe65 and histone acetyltransferase Tip60. Science 2001; 293:115-20
32. Yamamoto M, Kiyota T, Horiba M, Buescher J L, Walsh S M, Gendelman H E, Ikezu T: Interferon-gamma and tumor necrosis factor-alpha regulate amyloid-beta plaque deposition and beta-secretase expression in Swedish mutant APP transgenic mice. Am J Pathol 2007; 170:680-92
33. Liao Y F, Wang B J, Cheng H T, Kuo L H, Wolfe M S: Tumor necrosis factor-alpha, interleukin-1beta, and interferon-gamma stimulate gamma-secretase-mediated cleavage of amyloid precursor protein through a JNK-dependent MAPK pathway. J Biol Chem 2004; 279:49523-32
34. O'Connor T, Sadleir K R, Maus E, Velliquette R A, Zhao J, Cole S L, Eimer W A, Hitt B, Bembinster L A, Lammich S, Lichtenthaler S F, Hebert S S, De Strooper B, Haass C, Bennett D A, Vassar R: Phosphorylation of the translation initiation factor eIF2alpha increases BACE1 levels and promotes amyloidogenesis. Neuron 2008; 60:988-1009
35. Berridge M J: Inositol trisphosphate and calcium signalling. Nature 1993; 361:315-25
36. Wei H, Xie Z: Anesthesia, calcium homeostasis and Alzheimer's disease. Curr Alzheimer Res 2009; 6:30-5
37. Splettstoesser F, Florea A M, Busselberg D: IP(3) receptor antagonist, 2-APB, attenuates cisplatin induced Ca2+-influx in HeLa-S3 cells and prevents activation of calpain and induction of apoptosis. Br J Pharmacol 2007; 151:1176-86
38. Eckenhoff R G, Johansson J S, Wei H, et al. Inhaled anesthetic enhancement of amyloid-beta oligomerization and cytotoxicity. Anesthesiology 2004; 101:703-9.
39. Xie Z, Dong Y, Maeda U, et al. The Common Inhalation Anesthetic Isoflurane Induces Apoptosis and Increases Amyloid beta Protein Levels. Anesthesiology 2006; 104:988-94.
40. Xie Z, Dong Y, Maeda U, et al. The inhalation anesthetic isoflurane induces a vicious cycle of apoptosis and amyloid beta-protein accumulation. J Neurosci 2007; 27:1247-54.

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1.-24. (canceled)

25. A method comprising administering an effective amount of a compound of formula (III) to a subject in temporal proximity to administering an anesthetic to the subject, wherein the formula (III) has the structure: 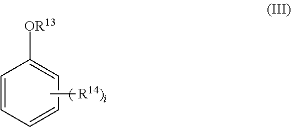
R₁₃ is selected from the group consisting of: hydrogen, CO₂R^{3A}, COR^{3A}, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl is optionally substituted;
R₁₄ is selected from the group consisting of: F, Br, Cl, I, CH₂NR^{3A}R^{4A}, SR^{3A}, SO₂R^{3A}, C₁-C₆ alkyl, C₁-C₆haloalkyl, C₁-C₆heteroalkyl, C₂-C₆alkenyl, C₂-C₆alkynyl, aryl or heteroaryl, NO₂, CF₃, or COR^{3A}, C(OH)R^{3A}, C(NOH)R^{3A}, C(S)R^{3A}, C(OH)(CF₃)R^{3A}, C(NOCH₃)R^{3A}, alkenyl wherein the alkyl, alkenyl, alkynyl, haloalkyl, heteroalkyl, heteroaryl, and aryl is optionally substituted;
R_{3A} and R_{4A} are each independently selected from the group consisting of: hydrogen, C₁-C₈ alkyl, C₂-C₈ alkenyl, C₂-C₈ alkynyl, C₁-C₈ haloalkyl, C₁-C₈ heteroalkyl, heteroaryl, or aryl, wherein the alkyl, alkenyl, alkynyl, haloalkyl, heteroalkyl, heteroaryl, and aryl is optionally substituted;
i=0, 1, 2, 3, 4, or 5;
or an enantiomer, prodrug, a derivative, or a pharmaceutically acceptable salt thereof.

26. The method of claim 25, wherein the compound of formula (III) is a 7-aminobutyric acid (GABA) receptor agonist.

27. The method of claim 26, wherein the compound of formula (III) is of formula (IV), wherein formula (IV) has the structure: 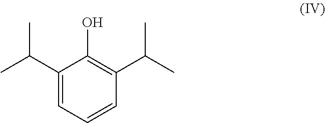
or an enantiomer, prodrug, a derivative or a pharmaceutically acceptable salt thereof.

28. The method of claim 25, wherein the compound is propofol, or an enantiomer, prodrug, a derivative or a pharmaceutically acceptable salt thereof, and the anesthetic is isoflurane.

29. The method of claim 25, wherein the subject is determined to be at risk for post-operative cognitive dysfunction (POCD) after administration of the anesthetic.

30. The method of claim 25, wherein the subject is diagnosed with or determined to be predisposed to a neurodegenerative disorder.

31. The method of claim 30, wherein the neurodegenerative disorder is Alzheimer's disease.

32. (canceled)

33. (canceled)

34. The method of claim 25, wherein the compound and the anesthetic are administered to the subject within one hour of each other.

35. The method of claim 25, wherein the compound is administered to the subject prior to the anesthetic.

36. The method of claim 25, wherein the compound is administered to the subject concurrently with the anesthetic.

37. The method of claim 25, wherein the effective amount is sufficient to decrease cognitive impairment in the subject resulting from the anesthetic by at least about 20%, as compared to absence of administration of the compound.

38. The method of claim 25, wherein the effective amount is sufficient to decrease a level of amyloid-β in a tissue of the subject resulting from the anesthetic by at least about 20%, as compared to absence of administration of the compound, or is sufficient to decrease apoptosis in a tissue of the subject resulting from the anesthetic by at least about 20%, as compared to absence of administration of the compound, or a combination thereof.

39. (canceled)

40. The method of claim 38, wherein the tissue is a brain tissue.

41. The method of claim 25, wherein the effective amount is from about 1 μM to about 1000 μM.

42. The method of claim 41, wherein the effective amount is in the range of about 10 μM to about 500 μM.

43. The method of claim 25, wherein the anesthetic is a halogenated ether anesthetic selected from the group consisting of: isoflurane, enflurane, halothane, sevoflurane and desflurane.

44. The method of claim 43, wherein the anesthetic is isoflurane.

45. The method of claim 25, wherein the subject is a mammal.

46. The method of claim 45, wherein the mammal is a human.

47.-78. (canceled)

79. A method comprising administering an effective amount of propofol, or an enantiomer, prodrug, a derivative, or a pharmaceutically acceptable salt thereof, to a subject in temporal proximity to administering isoflurane to the subject, wherein the subject is diagnosed with or determined to be predisposed to a neurodegenerative disorder.
